(19) United States
(12) Patent Application Publication
(10) Pub. No.: US 2024/0398724 A1
(43) Pub. Date: Dec. 5, 2024

(54) SKIN PATCH TEST FOR THE DIAGNOSIS OF IMMUNOLOGICAL PROTECTION AGAINST SARS-COV-2 VIRUS INFECTION

(71) Applicants: SEMMELWEIS EGYETEM, Budapest (HU); TERMÉSZETTUDOMÁNYI KUTATÓKÖZPONT, Budapest (HU); ORSZÁGOS KORÁNYI PULMONOLÓGIAI INTÉZET, Budapest (HU)

(72) Inventors: András FALUS, Érd (HU); Edit BUZÁS, Budapest (HU); Balázs SARKADI, Budapest (HU); Orsolya MÓZNER, Székesfehérvár (HU); Judit MOLDVAY, Budapest (HU)

(21) Appl. No.: 18/027,169

(22) PCT Filed: Sep. 20, 2021
(86) PCT No.: PCT/HU2021/050051

§ 371 Date: Mar. 20, 2023

(30) Foreign Application Priority Data
Sep. 18, 2020 (HU) P2000309

(51) Int. Cl. A61K 9/70; A61B 5/00; A61K 39/215

(52) U.S. Cl. CPC A61K 9/7084; A61B 5/411; A61B 5/6833; A61B 5/685; A61K 39/215; A61B 2562/12

(57) ABSTRACT

The invention relates to the application of a skin patch test for the detection of the specific cellular immune response against the SARS-CoV-2 (Severe Acute Respiratory Syndrome Coronavirus-2). The skin patch contains a formulated version of the recombinant form of SARS-CoV-2 antigenic protein. The application of the skin patch allows to follow the local skin reaction reflecting the strength of the cellular immune reaction against SARS-CoV-2, thus the skin test is applicable to a diagnostic evaluation of the specific cellular immunity evoked by previous virus infection or by vaccination against COVID-19.

### FIELD OF THE INVENTION

The invention relates to the application of a skin patch test for the detection of the specific cellular immune response against the SARS-CoV-2 (Severe Acute Respiratory Syndrome Coronavirus-2). The skin patch contains a formulated version of the recombinant form of the specific antigenic protein of the SARS-CoV-2 virus. The application of the skin patch allows to follow the local skin reaction reflecting the strength of the cellular immune reaction against SARS-CoV-2, thus the skin test is applicable to a diagnostic evaluation of the specific cellular immunity evoked by previous virus infection or by vaccination against COVID-19.

### BACKGROUND OF THE INVENTION

The antiviral immune response is based on the combined function of the cellular and humoral components. Currently the immune response against SARS-CoV-2 is preferentially examined by diagnostic tests measuring the presence of virus-specific IgM and IgG type antibodies in the blood plasma, secreted by the activated B cells. However, in the case of several viral diseases, including e.g. hepatitis C, AIDS, cytomegalovirus infection, as well as in some bacterial diseases, e.g. in tuberculosis (TBC), these antibodies do not provide an efficient protection against re-infection, and the basis of an efficient, long-term immunity is cell mediated immunity, CMI, provided by cellular components, especially by memory T cells. The multifunctional memory cell population (mainly T-CD4 and T-CD8 cells) is the key component of an active antiviral protection, because these cells can rapidly and more effectively develop specific cytokine release and cell killing responses, as compared to those provided by the naive immune cells (see Jaigirdar S A, et al. Development and Function of Protective and Pathologic Memory CD4 T Cells. Front Immunol. 2015. PMID: 26441961, Westerhof L M McGuire K, MacLellan L, Flynn A, Gray J I, Thomas M Goodyear C S, MacLeod M K. Multifunctional cytokine production reveals functional superiority of memory CD4 T cells. Eur J Immunol. 2019 November 49(11):2019-2029. doi:10.1002/eji.201848026).

There are by now numerous data in the literature showing a relatively small and inefficient humoral response to coronavirus infections, including also the SARS-CoV-2 infection. This response is hardly measurable in asymptomatic cases, and even if the specific antibodies appear in the blood, there is a rapid decline in the quantity of such antibodies (see Long Q X, et al. Clinical and immunological assessment of asymptomatic SARS-CoV-2 infections. Nat Med. 2020. PMID: 32555424, Braun et al, Presence of SARS-CoV-2-reactive T cells in 1 COVID-19 patients and healthy donors, 2020, MedRxiv doi: https://doi.org/10.1101/2020.04.17.20061440). In contrast, the cellular immune response in most cases is present and well measurable (Long Q X, et al. Clinical and immunological assessment of asymptomatic SARS-CoV-2 infections. Nat Med. 2020. PMID: 32555424, Braun et al, Presence of SARS-CoV-2-reactive T cells in 1 COVID-19 patients and healthy donors, 2020, MedRxiv doi: https://doi.org/10.1101/2020.04.17.20061440, Grifoni, A., et al, Targets of T Cell Responses to SARS-CoV-2Coronavirus in Humans with COVID-19 Disease and Unexposed Individuals. Cell 181, 1489-1501 (2020), doi: https://doi.org/10.1016/j.cell.2020.05.015).

For examining the cell mediated immunity (CMI) responses there are numerous techniques applicable in the diagnostics of bacterial, viral, or parasitic infections. In the detection of tuberculosis (TB) infection or previous immunization an often applied method is the Tuberculin Skin Test, TST, which can be carried out e.g. by the Mantoux method (intradermal injection of a purified protein derivative (PPD) of mycobacterium), or by multiple injections according to the MULTITEST CMI, from which the Mantoux method proved to be the most informative (Kasempimolporn et al., Application of transdermal patches with new skin test reagents for detection of latent tuberculosis. Journal of Medical Microbiology 2019; 68:1314-1319, DOI 10.1099/jmm.0.001037). Similar results were obtained for the detection of HIV infection (Martinez-Marcos, Francisco J. et al. Comparison of Two Methods for the Assessment of Delayed-Type Hypersensitivity Skin Responses in Patients with Human Immunodeficiency Virus Infection. Clinical Infectious Diseases 1998; 26:1330-1334). For detection of TB, Nakamura et al describes a method for skin patch test as well (Nakamura R M et al. Detection of active tuberculosis by an MPB-64 transdermal patch: afield study. Scand J Infect Dis. 2001; 33(6):405-7. Nakamura R M et al. MPB64 mycobacterial antigen: a new skin-test reagent through patch method for rapid diagnosis of active tuberculosis. INT J TUBERC LUNG DIS 1998, 2(7):541-546). Recently a TB skin test based on a microneedle patch test has also been disclosed (Wang, Wei et al. Skin test of tuberculin purified protein derivatives with a dissolving microneedle-array patch, Drug Deliv Transl Res 2019 August; 9(4):795-801. doi: 10.1007/s13346-019-00629-y.).

The CIM response in the skin test, based on the Mantoux method, is reflected by the local inflammation developing within 48-72 hours. This method cannot distinguish TB infection from the immunization by the BCG vaccine (see Kasempimolporn et al., Application of transdermal patches with new skin test reagents for detection of latent tuberculosis. Journal of Medical Microbiology 2019; 68:1314-1319, DOI 10.1099/jmm.0.001037), and may give a false positive reaction under certain conditions, e.g. viral or bacterial infections, stress reactions or metabolic diseases.

The DTH-type reactions are known to occur in several bacterial and viral diseases, thus their potential presence has been indicated for the identification of COVID-19 patients as well (C. S. Pavia and G. P. Wormser, COVID-19: Is there a role for Western blots and skin testing for determining immunity and development of a vaccine?, Diagnostic Microbiology & Infectious Disease (2020), https://doi.org/10.1016/j.diagmicrobio.2020.115148). In the case of HIV virus infection the skin test based on the DTH reaction was unstable and independent of the stage of the disease (Caiaffa W T et al. Instability of delayed-type hypersensitivity skin test anergy in human immunodeficiency virus infection. Arch Intern Med. 1995 Oct. 23; 155(19):2111-7. PMID: 7575072)

In the case of the SARS-1-coronavirus infection, strong DTH reaction and CD8⁺ CTL responses were observed in mice immunized with the nucleocapsid N protein (Zhao, Ping et al. Immune responses against SARS-coronavirus nucleocapsid protein induced by DNA vaccine. Virology. 2005 Jan. 5; 331(1): 128-135. doi: 10.1016/j.virol.2004.10.016, Zhao, Jincun et al., Identification and Characterization of Dominant Helper T-Cell Epitopesin the Nucleocapsid Protein of Severe Acute RespiratorySyndrome Coronavirus. J. Virol. 2007 Vol. 81, No. 11 p. 6079-6088 doi:10.1128/JVI.02568-06). In the case of the SARS-2-CoV infection, the analysis of the CD4⁺ T cell responses indicated that even in asymptomatic patients or in those suffering with mild symptoms, the T memory cell response was strong for several months. Since the early studies in the acute COVID-19 disease focused on the T cell responses in the period of lymphopenia, there are only limited information available for the T cell response in other tissues, e.g. in the lung (Stephens, D. S. and McElrath, M. J., JAMA, Sep. 11, 2020. doi:10.1001/jama.2020.16656). A study performed with a small number of patients reported the presence of CD4⁺ SARS-CoV-2-specific T-cells in 10 of 10 patients and in 8 of these patients the presence of specific CD8⁺ T-cells, with the strongest T cell responses against the Spike protein of the virus (Weiskopf D, Schmitz S S, Raadsen M P, Grifoni A, Okba N M A, Endeman H, et al. 2020. Phenotype and kinetics of SARS-CoV-2-specific T cells in COVID-19 patients with acute respiratory distress syndrome. Sci Immunol 48: eabd2071. doi: 10.1126/sciimmunol.abd2071).

In the current stage of studies there are numerous investigations attempting to decipher the strength and time-dependence of the CMI response in the COVID-19 disease and explore how efficient and sustained is the CMI-type protection. The first results in this regard indicate that after a SARS-CoV-2 infection virus-specific T cells appear, especially reacting with the cell surface proteins of this virus, predominantly with the Spike protein and its receptor binding domain (RBD) (Long Q X, et al. Clinical and immunological assessment of asymptomatic SARS-CoV-2 infections. Nat Med. 2020. PMID: 32555424, Braun et al, Presence of SARS-CoV-2-reactive T cells in 1 COVID-19 patients and healthy donors, 2020, MedRxiv doi: https://doi.org/10.1101/2020.04.17.20061440, Grifoni, A., et al, Targets of T Cell Responses to SARS-CoV-2 Coronavirus in Humans with COVID-19 Disease and Unexposed Individuals. Cell 181, 1489-1501 (2020), doi: https://doi.org/10.1016/j.cell.2020.05.015). The strongest and generally present response was observed against the Spike protein (Grifoni, A., et al, Targets of T Cell Responses to SARS-CoV-2 Coronavirus in Humans with COVID-19 Disease and Unexposed Individuals. Cell 181, 1489-1501 (2020), doi: https://doi.org/10.1016/j.cell.2020.05.015), while a non-specific cellular immune response, present in individuals not infected with SARS-CoV-2, was observed against other viral proteins, including the nucleocapsid (NP) and the non-structural (NSP-7 and NSP13) proteins as well. The potential explanation of this finding is that these latter proteins are well conserved among coronaviruses, thus a previous infection by these viruses might have caused a long-term cellular immune memory (see Le Bert, N., et al. SARS-CoV-2-specific T cell immunity in cases of COVID-19 and SARS, and uninfected controls. Nature (2020). https://doi.org/10.1038/s41586-020-2550-z, Stephens, D. S. and McElrath, M. J., JAMA, Sep. 11, 2020. doi:10.1001/jama.2020.16656). Based on the high immunogenicity of the Spike and the RBD proteins, serological tests have been developed, based on the production of these recombinant proteins in mammalian cells (Amanat F, et al. A serological assay to detect SARS-CoV-2 seroconversion in humans. Nat Med. 2020. PMID: 32398876).

According to the related news and other information, among the rapidly appearing diagnostic tests, methods for the detection of the cellular immunity against SARS-CoV-2 are also under development, for assessing the in vitro activation of blood-derived T cells (Med-Tech Innovation News, 2020 Sep. 1. https://www.med-technews.com/news/innovate-uk-backs-development-of-covid-19-t-cell-test/), including an ELISPOT/TSPOT test (Diagnostics from Technology Networks, 2020.07.08.: https://www.technologynetworks.com/diagnostics/blog/measuring-the-t-cell-immune-response-to-covid-19-337174j. All these sources emphasize the complicated nature of the cellular immunity studies (see Cookson C. et al., “T-cells: the missing link in coronavirus immunity?” Financial Times, 2020 Jul. 17. https://www.ft.com/content/5cf2ee49-df7a-4990-b337-860cf7737b2fj.

In the current state of technology, we did not find information about any skin-patch test for detecting cellular immune response in COVID-19 disease. There are information about a skin patch detecting the symptoms, e.g. fever or high pulse rate during virus infection (Sexton C., Smithsonian Magazine, 2020 Jul. 14, https://www.smithsonianmag.com/innovation/band-aid-patch-could-detect-early-covid-19-symptoms-180975301/), but this assay detects changes in coughing, breath frequency and pulse rate and no immunological reaction.

Skin patches are often applied for drug delivery. According to one method, virus antigens can be introduced into the deeper skin layers and this may serve as a vaccination method against SARS-CoV-2 infection (McKee S. PharmaTimes online 2020 Jul. 31. http://www.pharmatimes.com/news/uk_firm_starts_research_on_skin_patch_for_covid_vaccines 1346035).

In the present invention we disclose that a diagnostic evaluation of the CMI response against SARS-CoV-2 virus can be performed by a skin patch containing the antigenic Spike or RBD protein of this virus. The intradermal penetration of these proteins and the following DTH type reaction allows the estimation of the strength and persistence of the CIM response, and thus the immunological protection against this virus in the human patients.

### BRIEF DESCRIPTION OF THE INVENTION

The invention relates to a (trans)dermal skin patch, applicable for the detection of the cellular immune response against the SARS-CoV-2 virus in mammalian, preferably human patients, comprising:
an antigen-containing compartment or reservoir, containing a matrix and one or more recombinant SARS-CoV-2 antigen protein(s), recognizable by the cellular immune system,
a carrier, preferably a backing layer, carrying the antigen containing compartment,
attaching means for the fixing of the patch on the skin surface of the patient,
wherein, by fixing the skin patch on the surface of the skin of a patient, the one or more antigen protein(s) enter the epidermis of the human patient wherein said antigen protein(s) is/are contacted with the immune cells of the patient developed against the SARS-CoV-2 virus, and a local skin inflammation is evoked.

In an embodiment the dermal skin patch also comprises a reference compartment containing the matrix only and no SARS-CoV-2 antigen protein (i.e. does not comprise SARS-CoV-2 antigen protein).

In particular, the local skin inflammation is due to “Delayed-type hypersensitivity” (DTH).

Preferably the one or more recombinant SARS-CoV-2 antigen protein(s) comprise a spike protein of SARS-CoV-2 or an immunogenic part thereof. In a particular embodiment the recombinant SARS-CoV-2 antigen protein or the immunogenic part is the receptor binding domain (RBD) or an immunogenic part thereof.

In a preferred embodiment the SARS-CoV-2 antigen protein(s) comprise a surface epitope of the RBD. In a particular embodiment the surface epitope of the SARS-CoV-2 antigen protein(s) is a surface epitope exposed after cleavage by the TMPRSS2 serine protease.

In a particular embodiment the surface epitope of the SARS-CoV-2 antigen protein(s) is a surface epitope exposed before cleavage by the TMPRSS2 serine protease.

Preferably, in the dermal skin patch of the invention the attaching means is connected to the carrier or backing layer carrying the antigen containing compartment.

Preferably the attaching means comprises a carrier or backing layer and a glue for attaching said carrier or backing layer to the skin.

Preferably the carrier, preferably the backing layer of the attaching means and the carrier, preferably the backing layer carrying the antigen containing compartment are formed from the same continuous layer.

Preferably the matrix comprises excipient. Preferably the excipient and the matrix are dermatologically tolerable and preferably immunologically tolerable, highly preferably the excipient and the matrix do not evoke an immune response or (preferably) do not evoke an inflammatory immune response, in particular do not evoke inflammation upon contacting immune cells.

In an embodiment the invention relates to a kit of dermal skin patches, said kit comprising
a dermal skin patch comprising the antigen-containing compartment or reservoir, containing the matrix (with excipient) and one or more recombinant SARS-CoV-2 antigen protein(s) recognizable by the cellular immune system, and
a reference dermal skin patch comprising the reference compartment comprising the matrix and no SARS-CoV-2 antigen protein (i.e. does not comprise SARS-CoV-2 antigen protein) wherein
both the dermal skin patch comprising the antigen-containing compartment or reservoir and the reference dermal skin patch comprise
attaching means to fix the preparation on the skin surface of the human patient.

Preferably the dermal skin patch is called a transdermal skin patch. Preferably the skin patch is a dermal diagnostic skin patch. Preferably the skin patch is a transdermal diagnostic skin patch.

In an embodiment the skin patch contains more than one antigen containing compartments, each containing a given SARS-CoV-2 virus antigen protein.

In an embodiment the reference compartment contains, in addition to the matrix (excipient) a reference material e.g. a reference protein, which is non-antigenic.

In an embodiment the skin patch contains a further reference compartment, in which, in addition to the reference compartment comprising the matrix or the matrix only, the matrix (excipient) contains a reference material e.g. a reference protein, which is non-antigenic.

In an embodiment the kit comprises a further reference dermal skin patch, in which, in addition to the reference compartment comprising the matrix or the matrix only, the matrix (excipient) contains a reference material e.g. a reference protein, which is non-antigenic.

Preferably, the terms compartment and reservoir can be used interchangeably.

Preferably, in the skin patch according to the invention the one or more antigen proteins of the SARS-CoV-2 virus is of high antigenicity, including the isolated and purified Spike protein and/or its highly antigenic fragments with retained structure for preserved antigenicity.

Preferably, the isolated and purified Spike protein or immunogenic fragment thereof does not contain a natural proteolytic (furin or TMPRSS2) cleavage site.

Preferably, said protein or fragment does not contain the furin cleavage site.

Preferably, said protein or fragment does not contain the TMPRSS2 cleavage site.

Preferably, the isolated and purified Spike protein or its antigenic fragment is produced from among the groups of eukaryotic cells, including mammalian cells and insect cells.

Preferably, the isolated and purified Spike protein or its antigenic fragment is produced by a secretion pathway in the cells.

In a preferred embodiment the antigenic fragment of the recombinant SARS-CoV-2 virus Spike protein is the receptor binding domain (RBD) of the Spike protein.

In an embodiment the antigen protein is selected from the group consisting of the membrane protein (M), the spike protein (S) and the nucleoprotein (N) of SARS-CoV-2 virus or an antigenic (immunogenic) fragment thereof, in particular the receptor binding domain (RBD) of the spike protein.

In an embodiment the skin patch according to the invention comprises multiple antigen-containing compartments, each containing a recombinant SARS-CoV-2 antigen protein(s), recognizable by the cellular immune system. Preferably said antigen proteins are selected from the M, N, S proteins and the RBD of SARS-CoV-2 virus and (immunogenic) fragments thereof.

In a preferred embodiment the isolated and purified Spike protein or its antigen fragment is functionally pure with retained antigenicity, and the purity of the protein is at least 80%, preferably 90%, more preferably 95%, or even 98% or 99%, as compared to the amount of the total protein content in the preparation measured by accepted methods for measuring protein concentration, including e.g. a Bradford-assay, Coomassie Brilliant Blue G-250 staining, UV-absorption, bicinkonin acid method or Lowry method.

Very preferably the skin patch according to the invention contains one or more antigen proteins of the SARS-CoV-2 virus, present in extracellular vesicles (EV or ECV), while preserving the protein structure for antigenicity.

According to one preferred method the skin patch according to the invention contains one or more recombinant antigen proteins of the SARS-CoV-2 virus formulated by formulation means, supporting and promoting the entry of the antigen proteins into the epidermis, and thus allowing the induction of the local immune response, activation of the local antigen presenting cells and transfer the antigen to the regional lymph nodes. In this case, if the T memory cells specific for the SARS-CoV-2 virus antigens are present in the human patient, these cells will generate a macroscopic delayed type hypersensitivity (DTH) reaction, a local skin inflammation within 24-48 hours.

In the skin patch according to the invention preferably the antigen containing compartment and the reference compartment are localized in properly separated areas. In an embodiment the two compartments are localized in neighbouring areas.

In one preferred embodiment the matrix contains a gauze patch or a glass fibre patch, on which the one or more recombinant SARS-CoV-2 protein(s) are applied.

In a further preferred embodiment, the matrix contains a hydrogel, and the one or more recombinant SARS-CoV-2 protein(s) are provided within the gel.

In a preferred embodiment the skin patch comprises a permeation enhancer means for promoting or increasing the introduction of the one or more recombinant SARS-CoV-2 protein(s) into the epidermis. In a preferred embodiment of the application of the skin patch, the surface of the skin patch contains a further layer contacting the skin surface for promoting or allowing efficient introduction of the SARS-CoV-2 protein(s) into the epidermis. Preferably, this permeation enhancer means promotes the entry of each of the antigenic proteins into the epidermis.

One particular method of application is the use of a skin patch containing microneedles to promote the entry of the antigenic proteins. Preferably, the microneedles reach into epidermis, under its upper stratum corneum, or into the dermis.

The length of the microneedles is at least 20 μm up to about 3 mm, preferably 50 μm up to about 1 mm. In particular embodiments the length of the microneedles is as disclosed hereinbelow.

Another particular method of application is the use of a skin patch which contains one or more antigen proteins present in EV, while preserving the protein structure for antigenicity.

In a very preferred method of the invention the antigenic proteins are present in EVs and microneedles are used to introduce them to the epidermis.

In addition, the invention discloses a method for the preparation of a diagnostic skin patch to detect the cellular immune response against the SARS-CoV-2 virus in a human patient, characterized by the following:
i) preparation of one or more recombinant SARS-CoV-2 virus antigen proteins, preserving their native structure and antigenicity,
ii) providing a carrier or backing layer,
iii) on the carrier or backing layer the preparation contains
a. an antigen containing compartment containing a matrix (excipient) and one or more recombinant SARS-CoV-2 antigen protein(s), recognizable by the cellular immune system,
   and
b. a reference compartment containing the matrix (excipient) but no SARS-CoV-2 antigen proteins,
iv) the carrier or backing layer of the preparation is completed with means to fix the skin patch to the skin surface of the patient, wherein preferably, by attaching the skin patch to the skin surface of the patient, the antigen protein enters the epidermis, where it is able to contact the patient's immune cells against SARS-CoV-2 and cause inflammation of the skin.

In an embodiment of the method multiple antigen-containing compartments are formed on the carrier, each containing a recombinant SARS-CoV-2 antigen protein(s), recognizable by the cellular immune system.

Preferably said antigen proteins are selected from the M, N, S proteins and the RBD of SARS-CoV-2 virus and (immunogenic) fragments thereof.

Preferably said one or more proteins are recombinantly produced.

Preferably, the one or more SARS-CoV-2 antigen proteins are the recombinant Spike-protein and/or one or more antigenic fragments of the Spike-protein. Preferably, the recombinant Spike-protein lacks the natural (furin) cleavage site.

Very preferably, the receptor binding domain (RBD protein) is produced as an antigenic fragment of the Spike protein.

According to a preferred embodiment of the invention
during step i)
   the recombinant Spike-protein and/or one or more antigenic fragments thereof are expressed in eukaryotic cells selected from the following group:
      mammalian cells,
      insect cells,
   in secreted form, where the protein is secreted to the culture medium,
   the protein or protein fragment is isolated and purified from the cell culture medium using a tag to facilitate purification, while preserving the native structure required for antigenicity.

According to a preferred embodiment

the recombinant Spike protein and/or one or more antigenic fragments thereof is tagged to facilitate purification, preferably with a His-tag, and
the protein or protein fragment is isolated and purified from the cell culture medium, while preserving the structure required for antigenicity.

According to a preferred embodiment

the recombinant Spike protein and/or one or more antigenic fragments thereof are expressed in extracellular vesicles (ECV), while preserving the structure required for antigenicity.

Very preferably, the extracellular vesicles are prepared from insect or mammalian cell culture, preferably by TFF (tangential flow filtration) or ultrafiltration, and then isolated by size (optimally by size exclusion chromatography).

Preferably, the antigen carrier compartment and the reference compartment are created in a separate area on the carrier layer. The separate areas are preferably located next to each other. If more than two antigen-carrying compartments are needed, they are also created as separate areas.

Preferably, the antigen carrier compartment is formed by attaching a glass fibre layer as a matrix to the carrier layer, on which the one or more recombinant SARS-CoV-2 antigen proteins are dried. Very preferably, the fiberglass layer is a filter disc.

Alternatively, the antigen carrier compartment is formed by using a hydrogel as a matrix, into which the one or more recombinant SARS-CoV-2 antigen proteins are added, and the hydrogel is applied on the carrier layer,
alternatively, the application of the proteins in hydrogel is followed by gentle drying.

The separation of the compartment areas is also important here.

Very preferably, when the patch is applied, an additional layer in contact with the skin surface is formed on the skin-contacting surface of the patch, to deliver the SARS-CoV-2 antigen protein to the epidermis. In a special implementation, microneedles penetrating the epidermis are applied in the additional layer in contact with the skin surface. Preferably, the microneedles reach into epidermis, under its upper stratum corneum, or into the dermis.

The length of the microneedles is at least 20 μm up to about 3 mm, preferably 50 μm up to about 1 mm. In particular embodiments the length of the microneedles is as disclosed hereinbelow.

Another object of the invention is a diagnostic method for detecting the cellular immune response against SARS-CoV-2 virus in a human patient, comprising:
a patch according to any of claims 1 to 11 or any embodiment of the invention is applied to the skin surface of a human patient,
at least 12 or 24 hours, very preferably about 48 hours after application of the patch, the patch is removed, and
after removal of the patch, at least 36 or 48 hours, preferably 60 to 80 hours, very preferably 72 hours after application, the extent of the inflammatory skin reaction is observed in the skin area corresponding to the location of the antigen-carrying compartment and the skin area corresponding to the location of the reference compartment,
comparing the rate of inflammatory skin reaction in the skin area corresponding to the location of the antigen-carrying compartment and the skin area corresponding to the reference compartment, and
if the rate of inflammatory skin reaction in the skin area corresponding to the location of the antigen-carrying compartment exceeds the rate of inflammatory skin reaction in the skin area corresponding to the reference compartment, the presence of a cellular immune response against SARS-CoV-2 virus in the human patient is determined.

### Preferably

during the observation step, the skin area corresponding to the location of the antigen-carrying compartment and the skin area corresponding to the location of the reference compartment are photographed, and
the comparison is made on the basis of the picture.

According to a preferred embodiment
the image is analysed with a computer software method, by which the extent of the inflammatory skin reaction is quantified and assigned a numerical value.
the comparison is made by comparing the values assigned to the skin reactions observed in the skin area corresponding to the location of the antigen-carrying compartment and in the skin area corresponding to the reference compartment.

In the diagnostic procedure, preferably
if the difference in the degree of inflammatory skin reaction in the skin area corresponding to the reference compartment exceeds a predetermined standard value, then it is assessed that the human subject has a protective immune response.

Preferably, the patch is a microneedle patch and upon application of the patch the compartment(s) are pressed to introduce or inject the matrix into the epidermis or into the dermis.

In an embodiment the skin is slightly scraped before application.

Any of the above defined applications of one or more SARS-CoV-2 proteins in a structure that has native antigenic properties, preferably in a patch application according to the invention, is a subject-matter of the invention.

Preferably, the subject of the invention is an isolated and purified Spike protein and/or one or more antigenic fragments of it, where preferably, the isolated and purified Spike protein lacks the natural (furin) cleavage site, in the diagnostic process according to the invention, preferably for application on the skin patch according to the invention. Preferably, the protein is produced according to any of the above-defined processes.

According to a preferred embodiment, the antigenic fragment of the recombinant SARS-CoV-2 virus protein is the receptor binding domain (RBD-protein) of the Spike protein in the diagnostic process according to the invention, preferably for application on the skin patch according to the invention.

According to an aspect with an alternative wording, the following is the subject-matter of the invention.
1. the production of a skin patch preparation for human use, in order to detect a cellular, complex immune response against the SARS-CoV-2 virus,
a. which skin patch contains a recombinantly produced specific antigen protein of the SARS-CoV-2 virus, properly formulated.
b. which skin patch contains a negative control containing only the formulating material of the applied recombinant protein on its surface.
c. which skin patch, applied on the appropriate skin surface, is suitable for the diagnostic evaluation of the cellular, complex immunity against SARS-CoV-2, based on the degree of the inflammatory skin reaction (compared to the negative control response).
1.a.1. Preferably, the recombinant SARS-CoV-2 viral protein used on the skin patch is the highly antigenic Spike protein, produced to have the structure required for antigenicity while not containing the natural (furin) cleavage site.
1.a.1.1. Preferably, the recombinant, SARS-CoV-2 virus isolated and purified Spike protein applied on the skin patch in a secretory form in mammalian cells, produced in a labelled (e.g., his-tagged) form that allows rapid isolation and purification from cell culture medium and preserves the structure necessary for antigenicity.
1.a.1.2. Preferably, the recombinant, SARS-CoV-2 virus isolated and purified Spike protein applied on the skin patch in a secretory form in insect cells, produced in a labelled (e.g., his-tagged) form that allows rapid isolation and purification from cell culture medium and preserves the structure necessary for antigenicity.
1.a.2. Preferably, the recombinant, SARS-CoV-2 virus isolated and purified protein is the high-antigenicity receptor binding domain (RBD) of the Spike protein, produced to have the structure necessary for antigenicity.
1.a.2.1. Preferably, the isolated and purified receptor binding domain (RBD) of the recombinant SARS-CoV-2 in a secretory form in mammalian cells, produced in a labelled (e.g., his-tagged) form that allows rapid isolation and purification from cell culture medium and preserves the structure necessary for antigenicity.
1.a.2.2. Preferably, the isolated and purified RBD of the recombinant SARS-CoV-2 in a secretory form in insect cells, produced in a labelled (e.g., his-tagged) form that allows rapid isolation and purification from cell culture medium and preserves the structure necessary for antigenicity.
1.a.3. Preferably, the recombinant, SARS-CoV-2 virus Spike or RBD protein applied on the skin patch is produced in mammalian cells in a way that allows the expression of the SARS-CoV-2 virus Spike or RBD protein in the extracellular vesicles (ECV), and the proteins preserve the structure necessary for antigenicity in the isolated ECVs.
1.a.4. Preferably, the SARS-CoV-2 virus Spike or RBD protein, recombinant, isolated and purified, or expressed in ECVs, applied on the skin patch, formulated in a way that it permanently preserves the structure required for antigenicity, while it may effectively enter the epidermis and thus is suitable for the induction of the local immune response.
1.b. Preferably, two samples are applied to the skin patch on two adjacent but well-separated areas, one containing the formulated recombinant SARS-CoV-2 protein or protein fragment and the other containing only the formulation material. The formulation promotes the entry of the recombinant antigenic proteins into the epidermis.
1.b.1. Preferably, for the formulation of viral antigens, the patch contains a glass fibre filter disc on which the recombinant proteins are dried.
1.b.2. Preferably, for the formulation of viral antigens, the patch contains hydrogel, which continuously keeps the proteins in solution and facilitates their entry into the epidermis.
1.b.3. Preferably, a skin patch containing microneedles penetrating the epidermis is used to formulate the viral antigens, significantly enhancing the delivery of the viral antigens to the epidermis.

### Definitions

Herein, “skin patch” or “transdermal skin patch” or “dermal skin patch” means a preparation or device of a layered composition that can be affixed or attached to a skin surface, preferably a human skin surface, preferably adhered, and that carries an active ingredient, preferably an immunoreactive agent, for contact of the biological material with skin or with biological material found in the skin. The skin patch contains the active ingredient in a separate compartment, which serves as a reservoir, usually in a matrix (“matrix”, “matrix filler”), which allows controlled release. The patch contains the compartment (reservoir) attached to a carrier layer. Typically, the patch may contain an additional layer, preferably a membrane, through which the active ingredient must pass in order to come into contact with the skin surface, and thus optionally the additional layer or membrane controls/regulates the release of the active ingredient. Optionally, the skin patch contains a permeation enhancer, enhancing entry into the skin.

Preferably, the attachment of the skin patch on the skin surface occurs by the means of adhesion, where the adhesive is attached to the carrier layer, e.g. by an adhesive applied to the area of the carrier layer to which surface no area containing the active ingredient is connected.

A difficulty with skin patches is that the surface of the skin serves as a significant barrier to the entry of the active ingredient.

“Delayed-type hypersensitivity” (DTH) is an immune response reaction based on cell-mediated immunity; the term includes a cellular, preferably macroscopically visible, immune response elicited after the entry of any antigenic substance (immunological active ingredient) into the epidermis, in which an observable and detectable local inflammatory reaction due to memory immune cells recognising the antigenic substance already present in the body occurs. Typically, the local antigen-presenting cells are activated, take up the delivered protein, and transport it to the regional lymph nodes where the DTH response develops. The cells involved in the DTH response are primarily CD4+ Th1-lymphocytes, CD8+T-lymphocytes, in addition to monocytes, macrophages, and/or natural killer cells.

“Active ingredient” herein means a substance to be used for the benefit of a person under treatment or examination (“patient”), preferably a human, which has such a biological effect in the living organism of the person that is appropriate to the intended use. Immune response agent or antigenic agent is an agent that elicits an immune reaction in a living organism.

“Reservoir” or “compartment” means a container confined to a particular space that is suitable for “storing” an active ingredient, i.e., localizing it to that space for a desired period of time. This may occur by means of a container surrounded by a barrier or wall, in which case the storage is realised by physical barriers, or by means of a matrix, in which case the storage is realised by chemical means, typically by secondary binding forces. The use of a container or a matrix does not exclude the use of the other, respectively.

“Matrix” herein means a substance with the function of containing another substance, preferably an active ingredient. In one embodiment, the matrix is solid and “porous”, i.e. it contains spaces (cavities) which are not filled with the substance of the matrix and is thus suitable for storing the other substance, preferably the active ingredient. In another embodiment, the matrix is a high viscosity solvent in which the other substance, preferably the active ingredient, is in a dissolved state, however, the high viscosity and secondary chemical binding forces present in the solution allow storage.

“Diagnostics” means a procedure to determine the patient's state of health (healthy or sick, or the extent of these) by means of observation. Preferably, the observation is accompanied by a step or steps of procedure during which a biological substance in or derived from the patient's body is contacted with an active ingredient, in this case a “diagnostic agent”, and the reaction after contact is observed. The result of the observation makes it possible to form an opinion on the patient's state of health. The result of the observation may be qualitative or quantitative.

“Comparison”—As used herein, the “comparison” of the results of two reactions, preferably immunological reactions, observed during the course of a diagnostic procedure includes the operation of determining the difference (or deviation) between the two results. During the comparison, a qualitative or quantitative assessment is assigned to the difference or deviation. The determination of the difference may concern e.g. which reaction results in a larger and/or more intense discolouration of the skin, the assignment of numerical values (levels) to these results, or the determination of the values (levels) or their proportion, supplemented, if necessary, by other mathematical methods, when required by the evaluation (calculation) necessary for the procedure.

As used herein, “a/an” is to be construed as an indefinite article unless otherwise indicated in the context; and, where the context so permits, the definite article ‘the’ also includes the plural, unless the context otherwise requires. Accordingly, where the context so permits, the term “a/an” means “one or more”.

The term “isolated” means altered by the “human hand”, relative to the natural state. If a particular molecule or composition occurs in nature, it is “isolated” if it has been altered and/or removed from its original environment.

The term “purified” includes, in addition to isolation, the proportion of the purified substance (e.g., active ingredient) in a given mixture (e.g., solution or matrix) relative to other substances, e.g. substance(s) interfering with the intended purpose (contaminants) or to the total amount of the substance. The aim of “purification”, as a process, is the separation of other substance(s) (contaminants) that interfere with a given purpose and the substance to be purified and the production of the purified substance in a way that is suitable for further use.

The term “comprising” or “containing” is to be construed as meaning that the meaning of these is not exclusive and allows the addition or participation of additional properties or procedural steps or ingredients to or in anything containing the properties or process steps or ingredients listed.

As used herein, the term “substantially comprises” or “substantially contains” includes mandatory properties or process steps or ingredients listed in a list, e.g. a claim, and does not exclude the inclusion of other properties or procedural steps or ingredients that do not have an effect on the substantial properties on the application, process, composition or any other substance. As used herein, the term “comprising” or “containing” may be replaced by “substantially comprising” or “substantially containing”, if necessary, without the addition of a new substance.

Selection from a list, such as “selected”, may be replaced by “selected from the group consisting of” if the practice so requires and, if the context so permits, the selection of one or more items from the list is to be included.

### A BRIEF DESCRIPTION OF THE FIGURES

FIG. 1: a map of transposon vectors p10-RBD-IRES2-EGFP (FIG. 1A) and p10-Spike-IRES2-EGFP (FIG. 1B)

FIG. 2A: green fluorescence of single cell-based cell line No. 5, which stably produces RBD protein

FIG. 2B: green fluorescence of single cell-based cell line No. 11 stably producing Spike protein, cultured in a 6-well plate in DMEM medium containing serum

FIG. 3A: Coomassie staining of protein samples purified on a nickel-Sepharose column from the supernatant of RBD and Spike protein-producing cells (DMEM medium containing serum) following SDS-polyacrylamide electrophoresis

FIG. 3B: image of Western blot film with anti-His primary HRP-conjugated secondary antibody with the same samples as on FIG. 3.A

FIG. 3C: purified Spike protein. Following Coomassie staining, it can be clearly seen that no other protein contaminants appear in addition to the Spike protein during elution

FIG. 4: Western blot performed with the Spike protein-specific primary antibody. It shows that the antibody recognises the purified Spike protein, but not the RBD, as expected.

FIG. 5: Measuring RBD reactivity in a specific ELISA

FIG. 6: Coomassie staining of RBD lyophilized protein samples following SDS-polyacrylamide electrophoresis

FIG. 7A: Schematic map of the construct allowing the production of COVID-19 Spike/RBD proteins in ECVs

FIG. 7B: Plasmid construct (used with the Sleeping Beauty transposon system) enabling the expression of RBD viral protein in ECVs, for use in HEK cell lines

FIG. 7C: HEK cells producing the stable expression of the SARS-CoV-2 virus RBD protein, blue and green fluorescence.

FIG. 8: Preliminary animal toxicology results, indicating no effect on the survival or the weight of the animals. The purified, recombinant Spike protein (20 μg in 100 μL PBS) was either applied IV, IP, or onto the skin.

FIG. 9A: Antibody rapid test result of the subject participating in the skin-patch study, indicating the presence of anti-CoV-2 antibodies in the serum of the subject.

FIG. 9B: Preliminary human skin patch applied on the arm of the subject—upper band: the skin patch containing 20 μg Spike protein; middle band: the skin patch containing 20 μg RBD protein; lower band: the skin patch containing only the formulation material.

FIG. 9C: Preliminary human skin patch skin reaction results, showing positive reactions at the sites of the application of the Spike protein and the RBD protein.

### DETAILED DESCRIPTION OF THE INVENTION

The subject of the invention is a skin patch for detecting the presence and the degree of the specific cellular immune response against the SARS-CoV-2 (Severe Acute Respiratory Syndrome Coronavirus-2), which contains a version of the recombinantly produced, strong, specific antigenic protein of the SARS-CoV-2 virus, in a manner suitable for application on the skin. The application of the skin patch allows to follow the local skin reaction reflecting the strength of the cellular immune reaction thus the skin test is suitable for extensive diagnostic testing of the specific cellular immune response against SARS-CoV-2.

In the novel preparation (device) according to the invention, to determine a specific cellular immune response against SARS-CoV-2 virus, recombinant proteins of SARS-CoV-2 virus, Spike (S) protein, which has been shown to be a potent antigen, and its receptor binding domain (RBD) protein are used. The recombinant, expressed, purified Spike protein, or its antigenic fragment, preferably the RBD, is applied to the carrier layer surface of a skin patch after formulation as an immunological active ingredient. A negative control containing the formulating material of the SARS-CoV-2 protein is also used on the surface of the skin patch. The formulating material serves as a matrix for the storage of the active ingredient.

In a preferred embodiment, the active ingredient is prepared in extracellular vesicles.

During application, the skin patch is preferably applied to the skin surface of the flexor side of the forearm. The entry of the active ingredient (e.g. the Spike protein and/or the RBD) facilitates the activation of the local antigen-presenting cells, which then take up the delivered protein, and transport it to the regional lymph nodes. In this case, if the T memory cells specific for the SARS-CoV-2 virus antigens are present in the human patient, these cells will commonly generate a macroscopic DTH reaction, a local skin inflammation within 24-48 hours.

Thus, contact for at least 12 hours, preferably at least 24 hours, typically at least 40 or 48 hours, is required for an immunological (DTH) response to occur. If the active substance enters the skin more effectively, the immune response may develop sooner. According to a very preferred embodiment, the degree of the inflammatory skin reaction characteristic of immunity against SARS-CoV-2 (redness and diameter of swelling) is evaluated at 72 hours, following the removal of the applied skin patch after 48 hours, comparing the result to the negative control response.

The superior antigenicity of the recombinant viral proteins produced in mammalian cell cultures to be used in the present invention is supported by our experimental results.

The delivery of active ingredients into the skin may be a significant problem. According to the invention, preferably, certain methods and formulation are used that facilitate the entry of the proteins into the epidermis.

The skin is fundamentally made up of three layers of tissue: epidermis, dermis, and subcutaneous tissue. According to the invention, the antigenic active ingredient primarily needs to be delivered to the epidermis, under its upper stratum corneum, or into the dermis. These are essentially hydrophobic environments, so this should be taken into account during delivery.

Different methods are available for delivery.

Delivery can be facilitated by lipid-based emulsion formulation. Nanoemulsions can be prepared e.g. with phase transition induced by high-pressure homogenisation, microfluidization or temperature (Escobar-Chávez J. J., Rodriguez-Cruz I. M., Dominguez-Delgado C. L., Diaz-Torres R., Revilla-Vázquez A. L. Recent advances in novel drug carrier systems. InTech; 2012. Nanocarrier systems for transdermal drug delivery; pp. 201-240).

Many have developed nanoemulsion systems for transcutaneous delivery (Ledet G., Pamujula S., Walker V., Simon S., Graves R. Development and in vitro evaluation of a nanoemulsion for transcutaneous delivery. Drug Dev Ind Pharm. 2014; 40:370-379).

Lopez et al. obtained particularly good results in eliciting T cell responses to protect against the virus (Lopez P. A., Denny M., Hartmann A.-K., Alflen A., Probst H. C. Transcutaneous immunization with a novel imiquimod nanoemulsion induces superior T cell responses and virus protection. J Dermatol Sci. 2017; 87:252-259).

Liposomes may also be carriers for delivery in the solution according to the present invention; preferably prepared using lipids similar to skin lipids (Ashtikar M., Nagarsekar K, Fahr A. Transdermal delivery from liposomal formulations—evolution of the technology over the last three decades. J Control Release. 2016; 242:126-140).

Compared to the above solutions, the use of extracellular vesicles provides an even more direct and efficient method of delivery and is advantageous for delivery to tissues and for eliciting an immune response to antigens.

In addition to the purified recombinant proteins, a method is used in which the SARS-CoV-2 virus antigen proteins are expressed in extracellular vesicles, thereby facilitating the entry and presentation of the antigens into the skin.

Using extracellular vesicles (ECV), antigens are processed and immunologically presented with high efficiency (Robbins and Morelli, 2014 Nat Rev Immunol. doi: 10.1038/nri3622, van der Meel et al., Extracellular vesicles as drug delivery systems: Lessons from the liposome field Journal of Controlled Release, 195, 10 2014, 72-85 doi; 10.1016/j.jconrel.2014.07.049, Wahlund, C. J. E., Güclüler, G., Hiltbrunner, S., Veerman, R. E., Näslund, T. I, & Gabrielsson, S. (2017). Exosomes from antigen-pulsed dendritic cells induce stronger antigen-specific immune response. Scientific REPOTTS|7: 17095|DOI.10.1038/s41598-017-16609-6). Due to their high immunogenic potential, ECVs that contain recombinant viral antigens also form a promising cell-free system for generating a cell-mediated immune response. Thus, in one variation of the preparation, the SARS-CoV-2 antigens are applied to the skin patch in such a form.

Based on their size, the extracellular vesicles can be divided into three groups, although there is a discrepancy in the literature on the specification of dimensions: (i) small extracellular vesicles (often exosomes of MVB origin), 50-150 nm in diameter, (ii) medium-sized extracellular vesicles (microvesicles or ectosomes), 100 nm-1 μm in diameter, and (iii) large extracellular vesicles (e.g. apoptotic bodies), >1 μm in diameter (Théry, C., et al., (2018). Minimal information for studies of extracellular vesicles 2018 (MISEV2018): a position statement of the International Society for Extracellular Vesicles and update of the MISEV2014 guidelines JOURNAL OF EXTRACELLULAR VESICLES 2018, VOL. 7, 1535750 https://doi.org/10.1080/20013078.2018.15357501. Based on their size, exosomes appear to be the most preferred means of delivery (Bunggulawa, E. J., Wang, W., Yin, T. et al. Recent advancements in the use of exo-somes as drug delivery systems. J Nanobiotechnol 2018 16, 81. https://doi.org/10.1186/s12951-018-0403-9).

Although extracellular vesicles are relatively new devices in drug delivery, there is a significant methodology that can be used according to the present invention. (Villa, Federico et al. Extracellular Vesicles as Natural, Safe and Efficient Drug Delivery Systems Pharmaceutics 2019, 11(11), 557; https://doi.org/10.3390/pharmaceutics11110557).

The use of exosomes is described in detail in Antimisiaris, Sophia G. et al., including the production methods referred to therein (Antimisiaris, Sophia G. et al. Exosomes and Exosome—Inspired Vesicles for Targeted Drug Delivery Pharmaceutics 2018, 10(4), 218; https://doi.org/10.3390/pharmaceutics10040218).

According to a preferred method, also presented in the below examples, the conditioned medium of recombinant mammalian cells used for protein expression is enriched in ECVs using filtration, e.g. ultrafiltration or TFF (tangential flow filtration). ECVs are then isolated and characterised by size exclusion chromatography and differential centrifugation. (See e.g. Théry, C., et al., (2018). Minimal information for studies of extracellular vesicles 2018 (MISEV2018): a position statement of the International Society for Extracellular Vesicles and update of the MISEV2014 guidelines JOURNAL OF EXTRACELLULAR VESICLES 2018, VOL. 7, 1535750 https://doi.org/10.1080/20013078.2018.1535750).

Differences between extracellular vesicles and liposomes as drug delivery devices are discussed by van der Meel, Roy et al. (van der Meel, Roy et al. Extracellular vesicles as drug delivery systems: Lessons from the liposome field. August 2014 Journal of Controlled Release 195 DOI: 10.1016/j.jconrel.2014.07.049).

For the preparation of the invention cell cultures stably producing recombinant (virus-free) SARS-CoV-2 viral proteins are maintained. The production, purification and formulation of the proteins are carried out under conditions suitable for preparing preparations for human use. Appropriate authorisation of preparations is obtained.

Materials and manufacturing methods may preferably be applied in case of, e.g. in the preparation of the patches according to the invention. Cited documents e.g. those teaching such methods of preparation and features of patches, e.g. microneedle patches, are incorporated herein by reference.

In one embodiment of the preparation, hydrogel is applied for the formulation of viral antigens, which continuously keeps the proteins in solution and facilitates their entry into the epidermis.

Any protein-carrying hydrogel may be suitable. The hydrogel is preferably one which is also suitable for carrying extracellular vesicles.

In one embodiment, the hydrogel is carbopol-based. Carbopol is a synthetic polymer that is made up of crosslinked carbomers. Because they are anionic in nature, a base material, e.g. triethanolamine should be used for proper neutralization.

The version containing the hydrogel or extracellular vesicles can be implemented e.g. as specified in the following publications: (Jain Shashank et al. Formulation and rheological evaluation of ethosome-loaded carbopol hydrogel for transdermal application Drug Development and Industrial Pharmacy Volume 42, 2016-Issue 8, Pages 1315-1324; DOI: 10.3109/03639045.2015.1132227; Mourtas S et al. The effect of added liposomes on the rheological properties of a hydrogel: a systematic study J Colloid Interface Sci. 2008 Jan. 15; 317(2):611-9. DOI: 10.1016/j.jcis.2007.09.070).

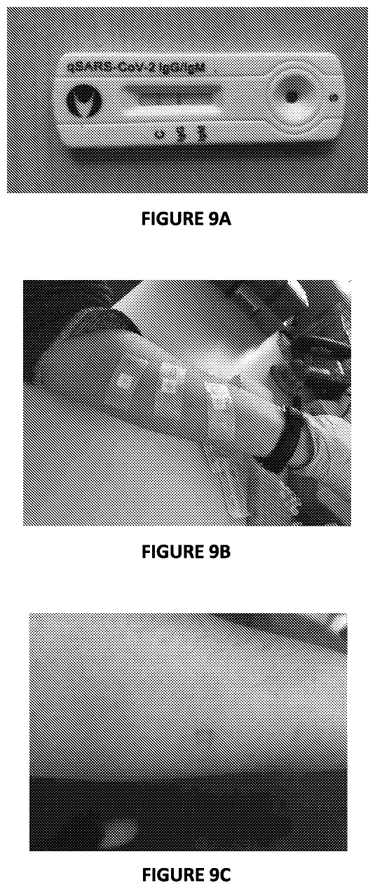

The present inventors have found that by introducing the antigens (e.g. either the spike protein or the RBD domain) e.g. by scraping (see FIG. 9) or in particular by a needle (100 μl), into the dermis (data not shown) elicits an immune response. In an embodiment the antigens are introduced into the epidermis. In a further embodiment the antigens are introduced into the dermis.

In another embodiment of the preparation, microneedles are used on the skin patch, that significantly increase the delivery of the viral antigens into the epidermis.

On microneedle patches (also called microneedle array patches or simply microarray patches) at least one or a plurality of microneedles is arranged.

Microneedles are micron-scale structures designed to pierce the skin, in particular the stratum corneum, and to permit delivery of an active ingredient to the epidermis or to the dermis (transdermal or intradermal delivery).

Microneedles reach epidermis, under its upper stratum corneum, or into the dermis. They never reach nerve endings and blood vessels. This means that the patients do not feel any pain when substances are delivered this way.

The length of the microneedles should be sufficiently large to penetrate the upper stratum corneum, but not so long as to penetrate the innervated part of the skin below the dermis. Thus, the microneedles may have a length of about at least 20 μm or 30 μm or 40 μm or 50 μm up to about 3 mm or 2 mm or 1 mm (1000 μm) or 500 μm or 200 μm. In a particular embodiment the length of the microneedle is 50 to 1000 μm. Microneedles may do not penetrate the dermis (in a preferred embodiment antigenic substances are delivered into the epidermis below the stratum corneum) and may have a length satisfying this criterion; e.g. the length may be less than about 500 μm or than about 400 μm. In more particular the length may be less than about 300 μm or than about 250 μm or less than about 200 μm. Thus, in a highly preferred embodiment the length of the microneedles is 50-500 μm or 50-300 μm or 50-200 μm.

In a further preferred embodiment the antigenic substances are delivered intro the dermis and may have a length satisfying this criterion; e.g. the length may be more than about 300 μm or more than about 250 μm or more than about 200 μm. In particular the length may be at least about 400 μm or at least about 500 μm. Also, in particular the length of the needles may be less than 3 mm, preferably at most 2 mm, more preferably at most about 1.5 mm or more particularly at most 1 mm or 1000 μm or 900 μm. Thus, in a highly preferred embodiment the length of the microneedles is 300 to 1500 μm or 500 to 1500 μm or 300 to 1000 μm.

Microneedle patches of various lengths are described e.g. in WO2016155891A1.

Microneedles may be solid microneedles, coated microneedles (for water soluble pharmaceutical formulations), dissolving microneedles or hollow microneedles (for liquid formulations). Solid microneedles are designed to produce relatively large pores in the skin. After the pores are formed, the matrix comprising one or more recombinant SARS-CoV-2 antigenic proteins or their antigenic fragments is delivered into the epidermis or into the dermis through the pores.

Hollow microneedles may also serve as antigen-containing compartment and contain the matrix comprising one or more recombinant SARS-CoV-2 antigenic proteins or their antigenic fragments a larger dose of a substance.

The material of the microneedles may be any number of materials, such as silicon, glass, polymer, biocompatible polymer, metal, ceramic, etc. The shape and sizes of the microneedles may vary according to the given application. The number of microneedles per unit area of the patch may also vary, typically between 2 and 100 microneedles per cm², but their density may be up to 5000 microneedles per cm² in high-density patches.

These are typically solid, drug coated solid or hollow microneedles.

Another option is to prepare dissolvable and swellable array of microneedles.

Various types of microneedles, like (i) solid microneedles, (ii) coated microneedles, (iii) dissolving microneedles, and (iv) hollow microneedles are described by Yang et al. (Yang et al. (2019). Recent advantages of microneedles for biomedical applications: drug delivery and beyond. Acta Pharmaceutica Sinica B, 9(3), 469-483.) The authors also teach materials and fabrication methods of microneedles.

According to an embodiment of the solution, microneedles can be formed from e.g. a hydrogel to pierce the stratum corneum of the epidermis. Such technology is described e.g. Mandal, A et al. They, however, intend to use the technology for sampling. By controlling the properties of the hydrogel and the properties of the matrix on the patch, as well as the flow conditions, with the help of a membrane, it is possible for the active ingredient to enter the epidermis. (Mandal, A et al. Cell and fluid sampling microneedle patches for monitoring skin-resident immunity. Science Translational Medicine, 14 Nov. 2018).

In one embodiment, for example, microneedles, more specifically a series thereof, can be formed by means of micromoulding as a means of enhancing penetration into the epidermis. According to one possibility, microneedles or spikes can be formed from e.g. hyaluronic acid gel in a polydimethylsiloxane (PDMS) mould form. The layer thus formed can be prepared as an additional layer of the patch in contact with the skin surface, which covers the antigenic active ingredient, i.e. the compartments containing the recombinant SARS-CoV-2 antigen proteins and the reference (Wang, Wei et al. Skin test of tuberculin purified protein derivatives with a dissolving microneedle-array patch, Drug Deliv Transl Res 2019 Augst; 9(4):795-801. doi: 10.1007/s13346-019-00629-y.).

In general, the manufacture of solid microneedle patches (arrays) is usually considered as a relatively simple approach and can be prepared e.g. from a variety of polymers including polycarbonate, polystyrene and polymethylmethacrylate (Martin, Alexander et al: Microneedle Manufacture: Assessing Hazards and Control Measures Safety 2017, 3, 25; doi:10.3390/safety3040025),

The application of microneedle patches is described as a pressing sensation and is essentially painless (Martin, Alexander et al., above).

The design of microneedle vaccines which are suitable to introduce antigens to the epidermis or the dermis, as described by Suh, Hyemee et al, “Microneedle patches for vaccine delivery” Clin Exp Vaccine Res 2014; 3:42-49 http://dx.doi.org/10.7774/cevr.2014.3.1.42.) When the patch is applied, it should be allowed to act for a sufficient time (see above). After removing the patch, the resulting skin lesion is analysed at the site of application.

The cellular immune response is inferred primarily from the diameter of the hyperaemic spot that appears.

The size of the spots can be calibrated based on clinical experience. The cellular response during the calibration is measured in patients who have proven cellular immune response or DTH reaction measured, as a reference, by a method other than that of the present invention. The data so obtained are compared and/or correlated with the properties of the skin lesion resulting from the DTH reactions elicited by the skin patch of the present invention, primarily the diameter of the spots and their colour intensity.

According to a preferred embodiment of the invention, a quantitative evaluation can also be carried out. During this, the affected skin area is photographed with specific size calibration (e.g., by placing a ruler or specifying the scale in software). As examples, the open source software products Imagej (FIJI) or Cell Profiler/Cell Analyst would be suitable for this. A compositive work could provide sufficient help for professionals in this regard (ld. pl. Aeffner Famke et al. Introduction to Digital Image Analysis in Whole-slide Imaging: A White Paper from the Digital Pathology Association. J Pathol Inform. 2019; 10: 9. doi: 10.4103/jpi.jpi_82_18).

A negative result according to the invention, i.e. when the spot corresponding to the antigen does not appear or does not differ significantly from the reference, indicates that the patient has not yet developed cellular immunity. A reason can be that the patient has not encountered the SARS-Cov-2 virus or has not developed immunity against the virus or due to vaccination or has lost the immunity e.g. due to the time passed. A clear positive result, on the other hand, would indicate that an adequate cellular immune response to the virus has been elicited by spontaneous immunization or active vaccination. In case of a positive result, it is worth performing further tests with other methods. In case of a negative result, appropriate measures should be taken in terms of protection against infection, vaccination etc.

If the result is doubtful, the diagnostic procedure should be repeated or an alternative detection of cellular immunity should be carried out.

For the diagnostic application of the composition, comparative clinical diagnostic studies are underway using the composition. In doing so, duly accredited clinical diagnostic organisations examine and evaluate the clinical picture, the level of disease onset, the time since recovery, and the presence of a humoral immune response (ELISA test) in parallel with the application of the skin patch preparation.

The patch can also be used to track the unique efficacy of SARS-CoV-2 vaccines.

The skin patch prepared as described herein or above can be stored and applied for a long time. The recombinant protein preparation does not contain components of SARS-CoV-2 virus other than one or more antigen(s) and thus cannot cause viral infection.

The diagnostic method that can be used with the help of the preparation is simple, widely used, and no significant side effects can be expected. The test performed using the skin patch formulation examines a complete local immune response in which a number of cell forms and tissue components are involved in the development of inflammation, i.e., it allows the estimation of a complex yet specific antiviral cellular immune response. The diagnostic method based on the preparation is suitable for measuring a cell-mediated immune response against SARS-CoV-2 that is variable in time and potency, but is expected to be more durable than the humoral immune response and better reflect actual immune protection. The preparation and the diagnostic method are suitable not only for monitoring direct immunisation after infection, but also for protection against possible re-infection and for evaluating the effectiveness of vaccination against SARS-CoV-2.

A significant advantage of the preparation of the invention lies in the fact that 1) the use of the specific antigenic protein, in particular the recombinant SARS-CoV-2 Spike/RBD antigen protein makes the determination of the cellular immune response against the virus specific, while the patch does not contain any other viral component, 2) the use and application of the skin patch does not require a separate laboratory infrastructure, can be widely used for mass monitoring, screening a large population, allowing it to be used as a “point of care” diagnostic method, 3) the skin patch preparation can be applied individually by non-professionals, e.g. the extent of the reaction can be read by a professional on the basis of an image transmitted by a mobile phone; in a preferred method, 4) the negative control on the skin patch formulation or kit allows reliable evaluation.

### EXAMPLES

The skin patch diagnostic test described in the present invention is designed for a simple and rapid assessment of the specific human cell-mediated immune reaction caused by the SARS-CoV-2 virus infection or the vaccination against this infection. The skin test contains the purified recombinant SARS-CoV-2 virus antigen, the Spike protein and/or its Receptor Binding Domain (RBD), formulated in formulation methods and materials to promote skin penetration into the epidermis.

The examples presented here provide the basis of a preclinical quality and safety documentation for the intended active substance and medicinal product. The investigational medicinal product in the examples includes two isolated and purified recombinant proteins, to be applied on the skin surface with limited penetration into the human body. Preclinical studies involving e.g. genotoxicity, in vivo metabolism, or elimination, are not relevant for this product.

### 1. Production of Isolated, Recombinant SARS-CoV-2 Spike and RBD Proteins

For recombinant Spike and RBD protein production the protein coding cassette of the DNA constructs presented in Amanat et. al (2020) (Amanat F, et al. A serological assay to detect SARS-CoV-2 seroconversion in humans. Nat Med. 2020. PMID: 32398876) were used. These sequences have the advantage of being codon-optimized for protein production in eukaryotic cells, and the sequences include a secretion signal peptide at the N-terminus that facilitates secreted protein production. It is also preferred that the protein sequences include a histidine-tag (his-tag) at the C-terminus, which allows for rapid isolation and purification. Amanat et. al shows that the DNA construct coding the Spike protein is designed to preserve the structure of the protein, while at the same time the naturally present furin cleavage that site disappears. Amanat et. al also shows that the protein structure and receptor binding function is preserved in the case of the RBD as well. Both proteins (Spike and RBD) were proved to preserve antigenicity.

The protein coding sequences of the original Amanat et al (2020) DNA constructs were used to create the plasmids that were designed to facilitate the stable expression of the Spike and RBD proteins in mammalian cells. The basis of this stable expression is the method described in Zámbó et. al (2020) (Zámbó B, Mózner O, Bartos Z, Török G, Várady G, Telbisz Á, Homolya L, Orbán T I, Sarkadi B. Cellular expression and function of naturally occurring variants of the human ABCG2 multidrug transporter. Cell Mol Life Sci. 2020 7(2):365-378. PMID: 31254042) where a plasmid was generated to achieve stable genomic integration in mammalian cells by using the Sleeping-Beauty transposon-transposase system and a fluorescent marker protein that helps to sort cells with the desired protein expression and create stable clonal cell lines.

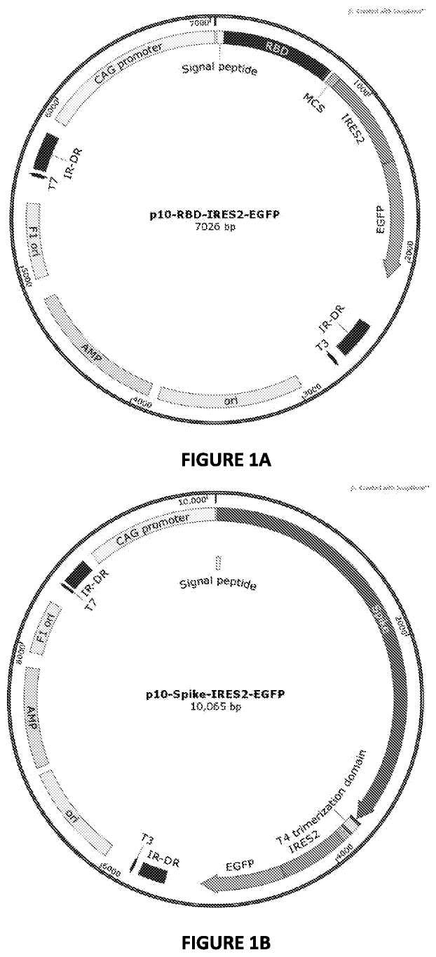

FIG. 1 shows the p10-RBD-IRES2-EGFP and p10-Spike-IRES2-EGFP transposon vector maps. In the transposon plasmids presented in FIG. 1, the regions between the IR-DR (inverted repeat/direct repeat element) sequences that integrate into the genome are the Spike or RBD coding sequences followed by an internal ribosome entry site (IRES2) and an EGFP (enhanced green fluorescent protein) coding sequence.

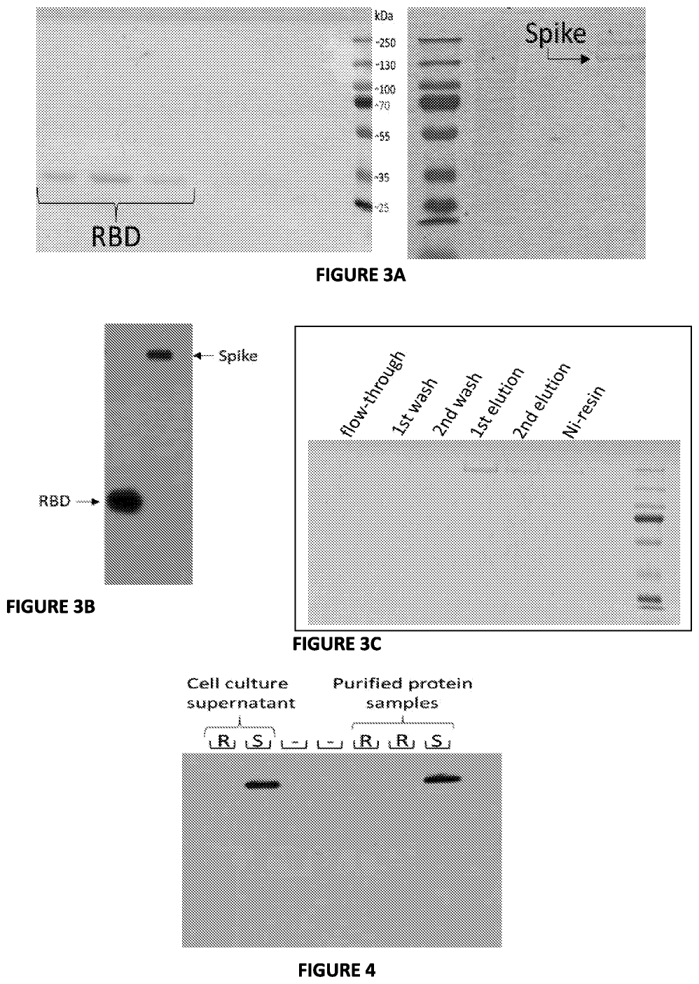

The commercially available human embryonic kidney (HEK-293-H) cell line was used for the expression of the recombinant SARS-CoV-2 modified Spike protein and the RBD protein. The p10-RBD-IRES2-EGFP and p10-Spike-IRES2-EGFP transposon DNA constructs were used for the transfection of the HEK-293-H cells, Lipofectamine 2000 was used for transfection. The successfully transfected HEK-293-H cells were sorted by BD FACS Aria II cell sorter based on the expression of the EGFP, and single-cell cloned on 96-well culture plates to generate clonal cell lines stably expressing high levels of the fluorescent protein. Several single cell-based cell lines were generated. Based on flow cytometry and confocal microscopy, cell line showing homogenous green fluorescence were selected for protein production. The high-level virus antigen (modified Spike or RBD) production in cell supernatants was examined by SDS-polyacrylamide gel electrophoreses and Western blotting. (FIG. 3A)

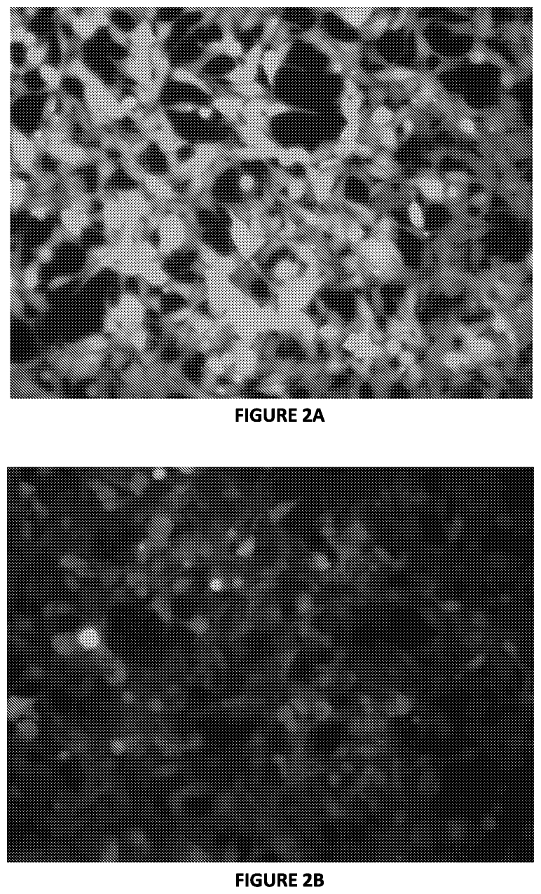

In the case of the Spike protein, about 10-fold lower expression was achieved compared to the RBD-expressing cell lines. This difference was observed in Amanat et. al (2020) as well, our theory is that it can be explained by the smaller size of the RBD protein. On FIG. 2 A, the green fluorescence can be seen of single cell-based cell line No. 5, which stably produces the RBD protein; on FIG. 2 B the green fluorescence can be seen of single cell-based cell line No. 11, which stably produces the Spike protein, cultured in 6-well cell culture plates and DMEM culture medium.

Cells were cultured in DMEM culture medium (Gibco™ DMEM, high glucose, GlutaMAX™), on 6-well plates, in T25, or T75 filter-cup cell culture flasks at 37° C. in an incubator with 5% CO2 containing air. The cells are passaged at 1-2×10⁶ cell/mL, twice a week at 1:10 ratio. The original and all following cell batches are stored in fluid nitrogen. Cell viability is assessed by Trypan blue staining.

The single cell-based cell lines were classified according to green fluorescence and the presence of the Spike or RBD protein was examined from the cell culture supernatant. The cell lines showing the highest expression of EGFP and the Spike or RBD protein were cultured on T25 cell culture flasks and have been transferred to serum-free chemically defined (Gibco™ FreeStyle™ 293 Expression Medium) culture medium, in which the cell culture became a suspension culture, and the lightly adherent cells became suspensible under a slight mechanical action. The FreeStyle culture medium designed for HEK-293 cell line-based serum-free protein production was used without any additional components. The cells were passaged at 1-2×10⁶ cell/mL, twice a week at 1:10 ratio (or with 1×10⁵ cell/mL seeding density).

The suspension cell cultures were also cultured as a shaken culture, thus achieving bigger cell density and better recombinant protein yield. 1 L Erlenmeyer cell culture flasks (Corning® Erlenmeyer cell culture flasks) with vented cap were used in an incubator on a shaker platform (100 rpm) and the cell seeding density was 2×10⁵ cell/mL, while 3-4×10⁶ cell/mL final cell density could be achieved. The supernatant is spun at 160×g for 5 minutes and stored at −20° C. until further processing.

The Spike and the RBD proteins, respectively, were isolated and purified by Ni-agarose chromatography. The stored supernatants are filtered with 0.2 μm pore size membrane filter, and optionally completed with 50 μg/ml (final concentration) protease inhibitor, PMSF. The purification is performed by using a column containing HisPur Ni-NTA Resin (88221, ThermoFisher), at room temperature or a HisTrap™ High Performance Ni Sepharose® column (GE17-5248-01, Sigma) on a fast protein liquid chromatography (FPLC) system.

The solutions used in the purification:

| Composition | Washing solution | Elution solution |
|---|---|---|
| Tris-HCl | 50 mM | 50 mM |
| NaCl | 300 mM | 300 mM |
| Imidazole | 10 mM | 200 mM |
| pH | 7.5 | 7.5 |

The steps of the purification:
1. Washing the column with washing solution
2. Application of the cell supernatant to the column
3. Incubation of the supernatant in the column
4. Washing the column with twice the volume of the washing solution
5. Elution by collecting fractions
6. Storage of the fractions at −80° C.
   Alternatively, PBS can be used instead of Tris-HCl, the purification was successful in both cases.

### Assays for Protein Purification:

The purified protein samples were examined after purification. The samples were separated by SDS-polyacrylamide gel electrophoresis, followed by Coomassie-blue protein staining and Western blot with anti-His primary and HRP-conjugated secondary antibodies. Total protein concentration was measured using the Qubit protein assay and calculated from the estimated extinction coefficient and absorbance observed at 280 nm on a NanoDrop 2000 Spectrophotometer. The Western blot detects the Spike and RBD protein in the sample, Coomassie staining gives information of all other protein that may be present in the sample in an amount comparable to the RBD and Spike protein (see FIGS. 3A1 and 3A2, respectively, shows Coomassie-blue protein staining following SDS-polyacrylamide gel-electrophoresis).

FIG. 3B shows a Western blot film of the purified RBD and Spike protein samples. Western blot development was performed with anti-His primary and HRP-conjugated secondary antibodies.

The samples were further purified and concentrated to eliminate imidazole from the solution. This washing step was performed 3 times with sterile PBS in Amicon Ultra 100K (Spike protein) or Amicon Ultra 30K (RBD) Centrifugal Filters.

The final recombinant protein product solutions were virus-inactivated by using UV irradiation (a 20 second UV light treatment—of 50 mJ/cm2). As documented in the relevant literature this UV treatment is sufficient to remove any kind of virus contamination in a solution (Lytle & Sagripanti, 2005; Thurston-Enriquez et al., 2003; Tseng & Li, 2005). This was performed by using an UV light source of 8 W, irradiation in solution of 10 mm depth, from 5 cm at room temperature for 20 seconds. This irradiation, up to 60 seconds, did not cause an aggregation of the purified protein.

The batch sizes to be used for further formulation are 1 mL of the protein solutions. To increase the shelf life of the protein samples, lyophilization was tested under different circumstances. Lyophilization was performed from 1 mg/ml, 0.5 ml protein solution under 0.1 mBar pressure at −60 to −40° C. for 48 hours. Different storing conditions were tested for the lyophilized samples.

### Cell Bank System, Characterization and Testing

As described above, we have generated several stable clones of the HEK cells expressing the Spike or the RBD protein in the supernatant. During all steps of cell-line generation we preserved frozen batches of the cells. For all quality, safety and clinical studies we have generated a main cell bank (MCB) consisting of 20 frozen vials (containing 1×10⁷ cells) of the final cell lines (Covicell-001-Spike and Covicell-001-RBD). All further studies refer to the production of the Spike or the RBD protein by using the cells obtained from this cell bank. The cells were characterized by the respective surface marker proteins. The correctness of the nucleotide sequences coding for the expressed proteins has been tested by sequencing.

All procedures were carried out under sterile conditions in level 2 safety laboratories and cabinets. Potential bacterial and virus contamination of the preparations was prevented by the culturing technology applied and any bacterial contamination was prevented by a final filtration step through 0.2 uM filters. Final inactivation of any potential human pathogenic viruses was achieved by the UV irradiation of the purified protein preparation, as described above.

For the stability and productivity of the recombinant clonal cell lines eGFP expression is an appropriate control, as eGFP expression is directly connected to recombinant virus protein production. For determining the concentration, structure and antigenic properties of the recombinant proteins we used several methods which can be used as controls of these steps.

### Characterization of the Protein Products

Less than 10% protein impurity is present in the final Spike or RBD protein solution. The purification protocol assures the removal of any DNA or RNA contamination. The potential proteins remaining in the preparation are originated from the host cell of human origin, and any virus contamination is removed during the last step, the UV irradiation of the purified protein solution. The remaining host cell protein contamination is not expected to affect the diagnostic application of the recombinant, purified protein preparations, applied on the human skin surface.

For the structure and basic characterization of the recombinant, purified Spike and RBD proteins see the examples above. The expected immunological activity, that is the specific antibody binding properties of the purified proteins have been examined in detailed immunological assays. In order to determine the immunological activity of these proteins we have performed several relevant assays:

A. Antibody recognition of the purified proteins on Western blot. This assay provides information about the specific recognition of the purified proteins after SDS polyacrylamide gel electrophoresis and immunoblotting by commercially available monoclonal antibodies. Since these conditions result in the denaturation of the purified proteins, positive recognition indicates that even the denatured protein is recognized by the specific monoclonal antibodies. As shown in FIG. 4, the isolated Spike protein (S) was recognized on the Western blot by the Spike-specific mouse monoclonal antibody (Abcam, cat. ab273433), the Spike protein was detectable in the cell culture supernatant and also in the purified protein sample. The anti-RBD rabbit monoclonal antibody (Abcam, cat. ab273074) did not recognize either the full-length Spike protein or the isolated RBD protein (blot not shown). To visualize the results, goat anti-mouse IgG (H+L) HRP conjugate (Abcam, cat. ab97023) and goat anti-rabbit IgG (H+L) HRP conjugate (Abcam, cat. ab6721) secondary antibodies were used. These data indicate that the anti-S mAb can be used even under denaturing conditions, while the anti-RBD mAb does not recognize the denatured RBD.

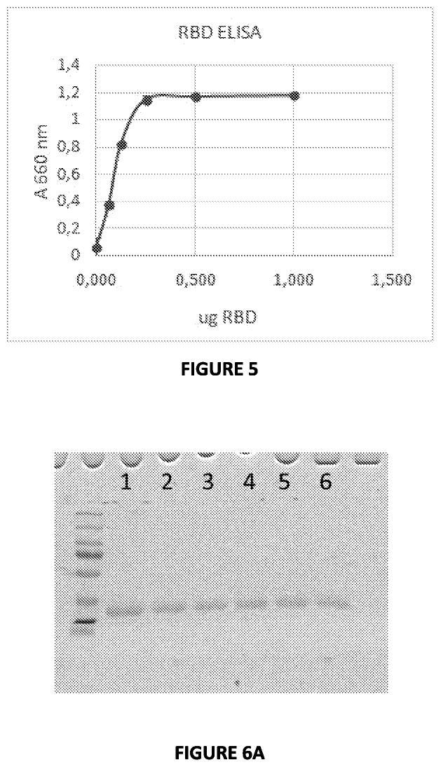

B. Antibody recognition of the purified proteins in ELISA. In this assay we have determined the specific recognition of the purified proteins in their native state by the anti-Spike and anti-RBD monoclonal primary and HRP-conjugated secondary antibodies (see FIG. 5). A chromogenic HRP-substrate was used, and samples were examined by a VictorX multilabel plate reader.

More closely, FIG. 5 illustrates the measuring of RBD reactivity in a specific ELISA. The RBD protein in PBS was dried (overnight at 4° C.) to wells in a 96 well plate. The samples were blocked by 0.5% BSA/PBS for one hour at room temperature, then washed 3× in PBS-0.1% Tween 20. The anti-RBD antibody (Abcam, cat. ab273074) was applied in 1:1500 dilution in 0.5% BSA/PBS for one hour at room temperature, then the wells washed 3 times in PBS-0.1% Tween 20. The secondary antibody (anti-rabbit HRP, Abcam, cat. ab6721) was applied in a dilution of 1:4000 in 0.5% BSA/PBS for 30 min, washed 3 times in PBS-0.1% Tween 20, then developed by using the TMB substrate+H2O2 (Thermo Scientific cat. 34021). The absorbance was read at 660 nm after 10 min.

C. Receptor binding assay in intact human cells carrying the ACE2 receptor. In this assay we have determined the specific binding of the recombinant, purified Spike and RBD proteins to human HepG2 cells expressing the ACE2 receptor on their cell surface. In these experiments we incubated the HEK cells with the solutions containing the Spike or the RBD proteins, and added the specific monoclonal antibodies mentioned in S.4.1.1. to these pre-incubated cells. Isolated protein binding was determined by using secondary, fluorescently labeled anti-mouse IgG, and measuring the fluorescence in flow cytometry.

D. Potential impurities, that is proteins remaining in the preparation originated from the host cell of human origin, should not pose any harmful effect in the intended use of the protein preparations. Any virus contamination is removed during the last step, the UV irradiation of the purified protein solution. The remaining host cell protein contamination is not expected to affect the diagnostic application of the recombinant, purified protein preparations, applied on the human skin surface.

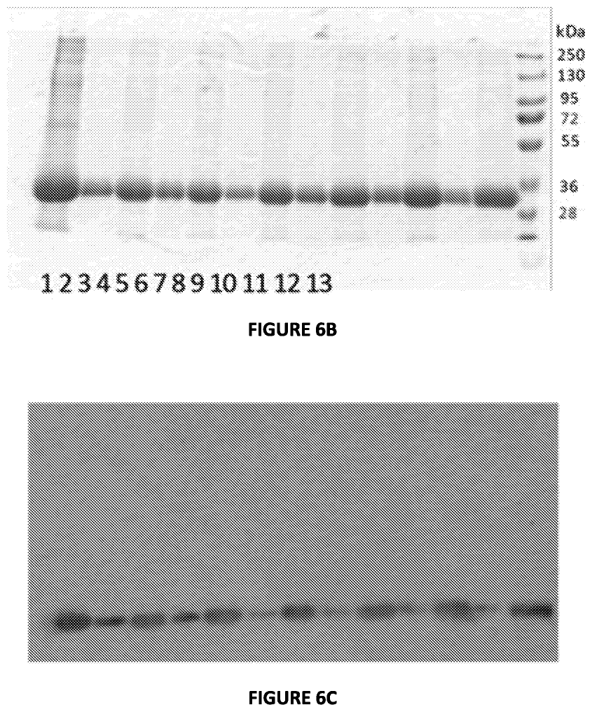

E. Lyophilization: For these studies we have used the purified RBD protein from samples frozen to −80° C. The effects of PBS and Tris buffer, as well as of sucrose (50 mg/ml), and trehalose (50 mg/ml) were studied, as shown in the table below. The lyophilized and frozen samples were dissolved in distilled water and run on a SDS-polyacrylamide gel, stained with Coomassie blue (FIG. 6)

The samples on FIG. 6A are listed as follows:

| Sample number | Buffer | Additional sugar |
|---|---|---|
| 1 | PBS | — |
| 2 | PBS | sucrose |
| 3 | PBS | trehalose |
| 4 | Tris-HCl | — |
| 5 | Tris-HCl | sucrose |
| 6 | Tris-HCl | trehalose |

The samples on FIGS. 6B and 6C are listed as follows

| | |
|---|---|
| 1 | 1 μg non-lyophilized RBD; 2020 12 |
| 2 | PBS- |
| 3 | PBS-80 |
| 4 | PBS-sucrose |
| 5 | PBS-sucrose-80 |
| 6 | PBS trehalose |
| 7 | PBS trehalose-80 |
| 8 | TRIS- |
| 9 | TRIS-80 |
| 10 | TRIS-sucrose |
| 11 | TRIS-sucrose-80 |
| 12 | TRIS-trehalose |
| 13 | TRIS-trehalose-80. |

The lyophilized samples showed very similar signals when examined by ELISA (anti-RBD antibody Abcam, cat. ab273074).

### 2. Recombinant RBD Protein Production by the Production of Extracellular Vesicles (ECVs)

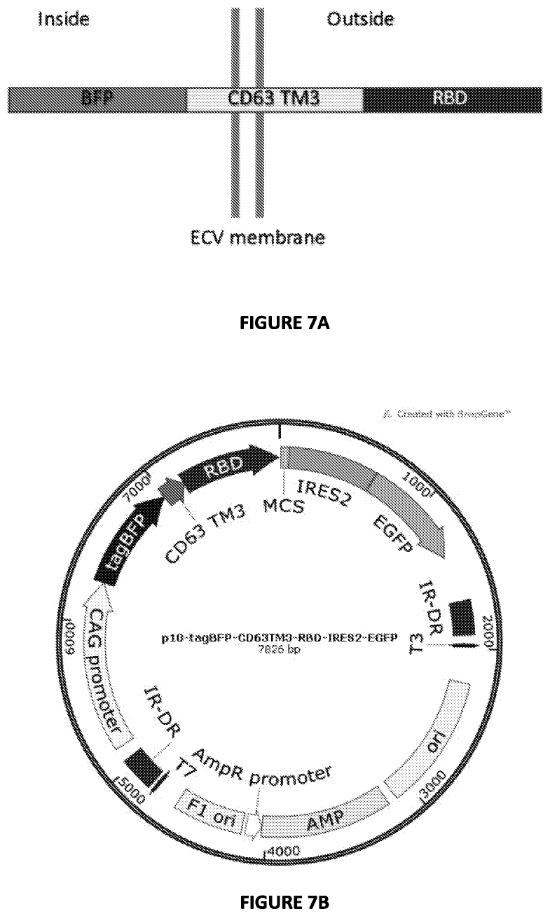

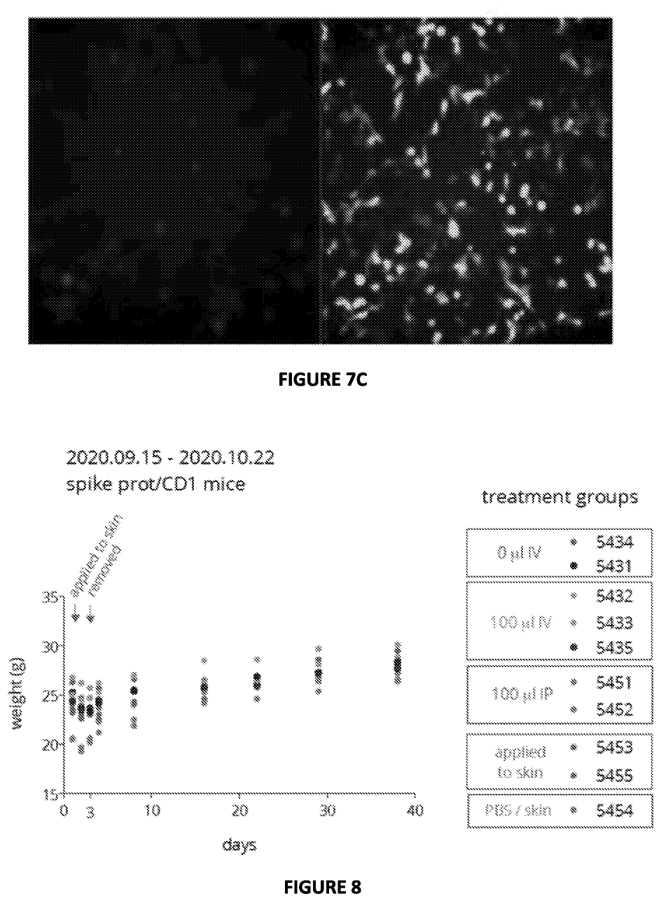

The proteins produced in extracellular vesicles are better absorbed by the antigen-presenting cells, thus they can more likely achieve an adequate immune response. Therefore, we express the recombinant virus-antigens in isolated ECVs as well. According to the relevant literature (Curley, N. et al., Sequential deletion of CD63 identifies topologically distinct scaffolds for surface engineering of exosomes in living human cells. Nanoscale, 2020, 12, 12014), one of the essential components in the ECVs is the 3th transmembrane domain (TM3) of the CD63 protein, which is sufficient for the formation and targeting of ECVs, while fusion proteins can be created on the N and C terminus of the TM3 domain (FIG. 7A). Accordingly, we have created the expression vector on FIG. 4B, that is expressed in HEK cells, thus ECVs that have the SARS-CoV-2 virus antigen (RBD) on their surface are produced, while a fluorescent marker (BFP) is expressed inside the vesicles.

On FIG. 7B the construct enabling stable expression of SARS-CoV-2 virus Spike or RBD protein on ECVs is illustrated. The construct enables the stable expression in HEK cells by the Sleeping Beauty transposon system, the cells stably expressing the ECVs with the SARS-CoV-2 virus antigens can be single-cell cloned. The vesicles that have the Spike or RBD is expressed on their surface show blue fluorescence. On FIG. 7C we present the results of using this expression vector, producing the stable expression of the SARS-CoV-2 virus RBD protein in HEK cells.

The HEK singe-cell based cell lines are cultured and cloned as described in Example 1, the ECVs are isolated and characterised according to the MISEV2018 guidelines (Théry, C., et al., (2018). Minimal information for studies of extracellular vesicles 2018 (MISEV2018): a position statement of the International Society for Extracellular Vesicles and update of the MISEV2014 guidelines JOURNAL OF EXTRACELLULAR VESICLES 2018, VOL. 7, 1535750 https://doi.org/10.1080/20013078.2018.1535750).

Briefly, the CD63 TM3 Spike/RBD fusion protein expressing HEK293 cells' conditioned media is filtered by TFF (tangential flow filtration) or ultrafiltration, thereby enriched in ECVs. Then the ECVs are isolated by size exclusion chromatography and differential centrifugation (2000 g→12500 g→100000 g). After isolation, followed by washing, the ECVs are characterised by immunogold EM (electron microscopy), TRPS (tuneable resistive pulse sending), NTA (Nanoparticle Tracking Analysis), micro BCA (bicinchoninic acid) and SPV (sulfo-phospho-vanillin) assays. High-resolution flow cytometry is also used to characterize the ECVs by detecting the blue fluorescence and the ECV-specific markers as well as detecting the Spike or RBD proteins by the specific anti-Spike or anti-RBD antibodies.

### 3. Formulation for the Use of the Recombinant, Purified SARS-CoV-2 Proteins Applied on a Skin Patch

To apply the recombinant proteins on the skin patch, the proteins are concentrated to 1 mg/mL in sterile PBS (and 0.02% Tween80) by ultrafiltration centrifugation (Amicon® Ultra Centrifugal Filters, Merck KGaA, Darmstadt, Germany).
3.a. In one example embodiment, 50 μg of recombinant protein is applied in 50 μL on a 5 mm diameter glass fibre filter (glass fibre filter, Whatman, Germany) and dried on the filter. As a negative control, 50 μL of PBS (and 0.02% Tween80) is used.
3.b In another example embodiment, the recombinant proteins (1 mg/mL) are applied in a hydrogel. This hydrogel corresponds to the already authorized and marketed gel EGIFERON, which is proved to be a non-toxic carrier (methyl parahydroxybenzoate, trolamine (triethanolamine), carbopol, water for injections).

### 4. Formulation for the Use of the ECVs Containing the Recombinant SARS-CoV-2 Proteins Applied on Skin Patch

According to a preferred embodiment, the SARS-CoV-2 recombinant proteins are expressed on the ECVs in a pre-loaded form described in Example 2.

The antigen-containing extracellular vesicles are mixed into Carbopol C974 hydrogel and formulated in the presence of parahydroxybenzoate and triethanolamine.

As an example, Carbopol 974P gel is carefully dispersed in distilled water or buffer (Hepes/NaCl) and parahydroxybenzoate and triethanolamine is added to the mixture. The pH of the mixture is adjusted according to the level the recombinant proteins require after the mixture is kept at 4° C. for 24 hours. Then the concentrated extracellular vesicles are added to the mixture, vortexed if necessary. The final carbopol concentration is 1-2 g/mL, for example 1.5 g/mL. The lipid concentration is adjusted to 2-10 nM. The dispersion is pH-adjusted and applied on the skin patch.

According to an alternative implementation, the final dispersion is dried on the carrier layer of the skin patch.

According to another implementation, the gel is transferred on the glass fibre filter disc already applied on the carrier layer of the skin patch.

During the diagnostic application, the patch is applied on the skin previously cleaned with alcohol-based cleaning solution. The patch is removed after 48 hours and the result is evaluated after 24 hours.

### Preclinical Safety Evaluation of the Protein Preparations Described in the Invention

The application of the skin patch test has no potential harmful effect in the human body, as 1) it contains only a purified recombinant protein and no virus or other potentially harmful biological component, 2) the formulation material is an already widely applied and accepted medicinal product, 3) the recombinant, purified virus antigen enters only the epidermis in the skin and has no systemic effect, 4) the local immune reaction has no potential harmful effect on human health, as testified by the wide-spread use of a similar diagnostic approach in tuberculosis immunization tests.

In order to assure the safety of the recombinant protein preparation, we have performed the following studies:

### In Vitro Assays:

A. We found no contamination by cellular DNA or RNA, as measured by the Qubit assay and direct cellular DNA determination.
B. Any potential live human pathogenic virus contamination was eliminated by the UV treatment as described above.

### In Vivo Assays:

### Preliminary Animal Toxicology Assay:

Mouse (CD1) toxicology experiments were performed at RCNS, Hungary. All treatments were performed under ZXBT narcosis. Experiments were performed under the conditions indicated in Table I.

TABLE I
Conditions of the preliminary toxicology experiments. Male CD1 mice
of the age of 5 weeks were treated as indicated in Table I.

| mouse | age (weeks) | weight (g) | sex | treatment |
|---|---|---|---|---|
| 5431 | 5 | 24.4 | m | none (mock TVI) |
| 5432 | 5 | 26.8 | m | 0.05 ug Spike prot IV (in 100 ul PBS) |
| 5433 | 5 | 25 | m | 0.05 ug Spike prot IV (in 100 ul PBS) |
| 5434 | 5 | 26.3 | m | none (mock TVI) |
| 5435 | 5 | 25.3 | m | 0.05 ug Spike prot IV (in 100 ul PBS) |
| 5451 | 5 | 20.5 | m | 0.05 ug Spike prot IP, in 100 ul PBS |
| 5452 | 5 | 20.6 | m | 0.05 ug Spike prot IP, in 100 ul PBS |
| 5453 | 5 | 23.5 | m | 17 ug Spike prot. in 100 ul PBS, applied to shaved skin |
| 5454 | 5 | 24 | m | 100 ul PBS applied to shaved skin |
| 5455 | 5 | 23.2 | m | 17 ug Spike prot. in 100 ul PBS, applied to shaved skin |

The purified Spike protein, dissolved in phosphate-buffered saline (PBS), in the amounts indicated, was administered intravenously in tail vene (IV), intraperitonally (IP), or in a skin patch on the shaved back skin of the mice. Skin patches were kept on for 72 hours, then removed. TVI: Tail Vene Injection. The conditions and the weight of the mice were followed for 43 days—see Table II.

TABLE II
Results of the preliminary toxicology experiments. Male CD1 mice
of the age of 5 weeks were treated as indicated in Table I.

| | weight (g) | | | | | |
|---|---|---|---|---|---|---|
| | 2020 Sep. 15 | 2020 Sep. 16 | 2020 Sep. 17 | 2020 Sep. 18 | 2020 Oct. 21 | 2020 Oct. 26 |
| mouse | day 1 | day 2 | day 3 | day 4 | day 38 | day 43 |
| 5431 | 24.4 | 23.5 | 23.3 | 24.5 | 28.4 | 28.5 |
| 5432 | 26.8 | 26.2 | 25.7 | 26.2 | 30.1 | 30.3 |
| 5433 | 25 | 24.5 | 24.7 | 25.3 | 28.9 | 29.4 |
| 5434 | 26.3 | 24.7 | 24.7 | 25.7 | 29.5 | 29.5 |
| 5435 | 25.3 | 23.8 | 23.7 | 24.2 | 27.7 | 28.4 |
| 5451 | 20.5 | 19.7 | 20.6 | 22.3 | 27.2 | 27.8 |
| 5452 | 20.6 | 19.3 | 20.2 | 21.2 | 26.6 | 27.3 |
| 5453 | 23.5 | 22.8 | 23 | 22.5 | 28.1 | 27.6 |
| 5454 | 24 | 23.1 | 23.5 | 23.7 | 26.4 | 25.9 |
| 5455 | 23.2 | 22.6 | 23.6 | 23 | 28.1 | 28 |

Adverse effects observed: mice 5431 and 5435: tail necrosis at the site of injection. The experiment was terminated at day 43 (Oct. 26, 2020) by narcosis of the mice. Blood samples were taken by heart puncture for further studies.
The representation of the data documented in Table II. can be seen in FIG. 8.

### Preliminary Human Skin Patch Assay

This preliminary assay has been performed by one of the medical doctors among the inventors, as a self-experiment. The patient has been vaccinated against SARS-CoV-2 by the Pfizer vaccine and the antibody rapid test showed a positive result for the presence of specific IgG (see FIG. 9A).

In the skin test assay the RBD preparation was used formulated as follows: 50 μg of recombinant protein was applied in 50 μL of PBS+0.02% Tween 80, on a 5 mm diameter glass fibre filter (glass fibre filter, Whatman, Germany) and immediately applied to the skin after mild scraping. As a negative control, 50 μL of PBS+0.02% Tween 80 was used. The preliminary test results are shown in FIGS. 9B and 9C.

The skin reaction was photographed removing the skin patches after 48 hours. The RBD protein-containing patch (and, slightly, the Spike containing patch) shows local immunoreaction, as compared to the control patch (FIG. 9C).

Further human studies are underway with GMP prepared preparations and with an ethical permission to involve human subjects.

### REFERENCES

Amanat F, et al. A serological assay to detect SARS-CoV-2 seroconversion in humans. Nat Med. 2020. PMID: 32398876
Aeffner Famke et al. Introduction to Digital Image Analysis in Whole-slide Imaging: A White Paper from the Digital Pathology Association. J Pathol Inform. 2019; 10: 9. doi: 10.4103/jpi.jpi_82_18
Antimisiaris, Sophia G. et al. Exosomes and Exosome-Inspired Vesicles for Targeted Drug Delivery Pharmaceutics 2018, 10(4), 218; https://doi.org/10.3390/pharmaceutics10040218
Ashtikar M., Nagarsekar K., Fahr A. Transdermal delivery from liposomal formulations—evolution of the technology over the last three decades. J Control Release. 2016; 242:126-140.
Bunggulawa, E. J., Wang, W., Yin, T. et al. Recent advancements in the use of exosomes as drug delivery systems. J Nanobiotechnol 16, 81 (2018). https://doi.org/10.1186/s12951-018-0403-9
Braun et al, Presence of SARS-CoV-2-reactive T cells in 1 COVID-19 patients and healthy donors, 2020, MedRxiv doi: https://doi.org/10.1101/2020.04.17.20061440
Caiaffa W T et al. Instability of delayed-type hypersensitivity skin test anergy in human immunodeficiency virus infection. Arch Intern Med. 1995 Oct. 23; 155(19):2111-7. PMID: 7575072)
Curley, N. et al., Sequential deletion of CD63 identifies topologically distinct scaffolds for surface engineering of exosomes in living human cells. Nanoscale, 2020, 12, 12014
Escobar-Chávez J. J., Rodríguez-Cruz I. M., Dominguez-Delgado C. L., Diaz-Torres R., Revilla-Vázquez A. L. Recent advances in novel drug carrier systems. InTech; 2012. Nanocarrier systems for transdermal drug delivery; pp. 201-240.
Grifoni, A., et al, Targets of T Cell Responses to SARS-CoV-2Coronavirus in Humans with COVID-19 Disease and Unexposed Individuals. Cell 181, 1489-1501 (2020), doi: https://doi.org/10.1016/j.cell.2020.05.015.
Jaigirdar S A, et al. Development and Function of Protective and Pathologic Memory CD4 T Cells. Front Immunol. 2015. PMID: 26441961,
Jain Shashank et al. Formulation and rheological evaluation of ethosome-loaded carbopol hydrogel for transdermal application Drug Development and Industrial Pharmacy Volume 42, 2016—Issue 8, Pages 1315-1324; DOI: 10.3109/03639045.2015.1132227
Kasempimolporn et al., Application of transdermal patches with new skin test reagents for detection of latent tuberculosis. Journal of Medical Microbiology 2019; 68:1314-1319, DOI 10.1099/jmm.0.001037.
Le Bert, N., et al. SARS-CoV-2-specific T cell immunity in cases of COVID-19 and SARS, and uninfected controls. Nature (2020). https://doi.org/10.1038/s41586-020-2550-z
Ledet G., Pamujula S., Walker V., Simon S., Graves R. Development and in vitro evaluation of a nanoemulsion for transcutaneous delivery. Drug Dev Ind Pharm. 2014; 40:370-379.
Long Q X, et al. Clinical and immunological assessment of asymptomatic SARS-CoV-2 infections. Nat Med. 2020. PMID: 32555424
Lopez P. A., Denny M., Hartmann A.-K., Alflen A., Probst H. C. Transcutaneous immunization with a novel imiquimod nanoemulsion induces superior T cell responses and virus protection. J Dermatol Sci. 2017; 87:252-259
Lytle, C. D., & Sagripanti, J.-L. (2005). Predicted inactivation of viruses of relevance to biodefense by solar radiation. Journal of Virology. 79(22), 14244-14252. https://doi.org/10.1128/JVI.79.22.14244-14252.2005
Mandal, A et al. Cell and fluid sampling microneedle patches for monitoring skin-resident immunity. Science Translational Medicine 14 Nov. 2018
Martin, Alexander et al: Microneedle Manufacture: Assessing Hazards and Control Measures Safety 2017, 3, 25; doi:10.3390/safety3040025
Martinez-Marcos, Francisco J. et al. Comparison of Two Methods for the Assessment of Delayed-Type Hypersensitivity Skin Responses in Patients with Human Immunodeficiency Virus Infection. Clinical Infectious Diseases 1998; 26:1330-1334.
McKee S. PharmaTimes online 2020 Jul. 31. http://www.pharmatimes.com/news/uk_firm_starts_research_on_skin_patch_for_covid_vaccines 1346035.
Med-Tech Innovation News, 2020 Sep. 1.
https://www.med-technews.com/news.innovate-uk-backs-development-of-covid-19-t-cell-test/, ELISPOT/TSPOT tesztrendszer; Diagnostics from Technology Networks, 2020.07.08.:
https://www.technologynetworks.com/diagnostics/blog/measuring-the-t-cell-immune-response-to-covid-19-337174
Mourtas S et al. The effect of added liposomes on the rheological properties of a hydrogel: a systematic study J Colloid Interface Sci. 2008 Jan. 15; 317(2):611-9. DOI: 10.1016/j.jcis.2007.09.070)
Nakamura R M et al. Detection of active tuberculosis by an MPB-64 transdermal patch: a field study. Scand J Infect Dis. 2001; 33(6):405-7.
Nakamura R M et al. MPB64 mycobacterial antigen: a new skin-test reagent through patch method for rapid diagnosis of active tuberculosis. INT J TUBERC LUNG DIS 1998, 2(7):541-546
Pavia C. S. and Wormser G. P., COVID-19: Is there a role for Western blots and skin testing for determining immunity and development of a vaccine?, Diagnostic Microbiology & Infectious Disease (2020), https://doi.org/10.1016/j.diagmicrobio.2020.115148.
Robbins and Morelli, 2014 Nat Rev Immunol. doi: 10.1038/nri3622,
Sexton C., Smithsonian Magazine, 2020 Jul. 14, https://www.smithsonianmag.com/innovation/band-aid-patch-could-detect-early-covid-19-symptoms-180975301/,
Stephens, D. S. and McElrath, M. J., JAMA, Sep. 11, 2020. doi:10.1001/jama.2020.16656
Suh, Hyemee et al, “Microneedle patches for vaccine delivery” Clin Exp Vaccine Res 2014; 3:42-49 http://dx.doi.org/10.7774/cevr.2014.3.1.42.
Théry, C., et al., (2018). Minimal information for studies of extracellular vesicles 2018 (MISEV2018): a position statement of the International Society for Extracellular Vesicles and update of the MISEV2014 guidelines JOURNAL OF EXTRACELLULAR VESICLES 2018, VOL. 7, 1535750 https://doi.org/10.1080/20013078.2018.1535750
Thurston-Enriquez, J. A., Haas, C. N., Jacangelo, J., Riley, K., & Gerba, C. P. (2003). Inactivation of feline calicivirus and adenovirus type 40 by U V radiation. Applied and Environmental Microbiology, 69(1), 577-582. https://doi.org/10.1128/aem.69. 1.577-582.2003
Tseng, C.-C., & Li, C.-S. (2005). Inactivation of Virus-Containing Aerosols by Ultraviolet Germicidal Irradiation. Aerosol Science and Technology, 39(12), 1136-1142. https://doi.org/10.1080/02786820500428575
van der Meel et al., Extracellular vesicles as drug delivery systems: Lessons from the liposome field Journal of Controlled Release, 195, 10 2014, 72-85 doi: 10.1016/j.jconrel.2014.07.049
Villa, Federico et al. Extracellular Vesicles as Natural, Safe and Efficient Drug Delivery Systems Pharmaceutics 2019, 11(11), 557; https://doi.org/10.3390/pharmaceutics11110557). (Wang, Wei et al. Skin test of tuberculin purified protein derivatives with a dissolving microneedle-array patch, Drug Deliv Transl Res 2019 August; 9(4):795-801. doi: 10.1007/si3346-019-00629-y.)
Wahlund, C. J. E., Güclüler, G., Hiltbrunner, S., Veerman, R. E., Näslund, T. I., & Gabrielsson, S. (2017). Exosomes from antigen-pulsed dendritic cells induce stronger antigen-specific immune response. Scientific REPOTTS|7: 17095|DOI:10.1038/s41598-017-16609-6
Weiskopf D, Schmitz S S, Raadsen M P, Grifoni A, Okba N M A, Endeman H, et al. 2020. Phenotype and kinetics of SARS-CoV-2-specific T cells in COVID-19 patients with acute respiratory distress syndrome. Sci Immunol 48: eabd2071. doi: 10.1 126/sciimmunol.abd2071)
Westerhof L M, McGuire K, MacLellan L, Flynn A, Gray J I, Thomas M, Goodyear C S, MacLeod M K. Multifunctional cytokine production reveals functional superiority of memory CD4 T cells. Eur J Immunol. 2019 November; 49(11):2019-2029. doi: 10.1002/eji.201848026. Epub 2019 Jun. 13. PMID: 31177549
Yang et al. (2019). Recent advantages of microneedles for biomedical applications: drug delivery and beyond. Acta Pharmaceutica Sinica B, 9(3), 469-483.
Zámbó B, Mózner O, Bartos Z, Török G, Várady G, Telbisz Á, Homolya L, Orbán T I, Sarkadi B. Cellular expression and function of naturally occurring variants of the human ABCG2 multidrug transporter. Cell Mol Life Sci. 2020 7(2):365-378. PMID: 31254042
Zhao, Ping et al. Immune responses against SARS-coronavirus nucleocapsid protein induced by DNA vaccine. Virology. 2005 Jan. 5; 331(1): 128-135. doi: 10.1016/j.virol.2004.10.016, Zhao, Jincun et al., Identification and Characterization of Dominant Helper T-Cell Epitopes in the Nucleocapsid Protein of Severe Acute Respiratory Syndrome Coronavirus. J. Virol. 2007 Vol. 81, No. 11 p. 6079-6088 doi:10.1128/JVI.02568-06)

1. Diagnostic skin patch for detecting the cellular immune response against a SARS-CoV-2 virus in a human patient, substantially comprising:
an antigen-containing compartment, containing a matrix comprising one or more recombinant SARS-CoV-2 antigenic proteins or their antigenic fragments,
a carrier layer that carries the antigen-containing compartment,
attaching means for the attachment of the patch on the skin surface of the patient,
wherein preferably, by attaching the skin patch on the skin surface of the human patient, the antigenic protein or their fragments enter the epidermis, where it is able to contact the patient's immune cells against SARS-CoV-2 and elicit an inflammatory immune response in the skin.

2. The diagnostic skin patch according to claim 1, also comprising a reference compartment, that contains the matrix but does not comprise SARS-CoV-2 antigenic protein(s).

3. A diagnostic skin patch according to claim 1, wherein the one or more recombinant SARS-CoV-2 antigenic protein highly antigenic, isolated and purified Spike protein and/or one or more antigenic fragments thereof, that preserved the structure requires for antigenicity.

4. A diagnostic skin patch according to claim 2, wherein the isolated and purified Spike protein lacks the natural cleavage site, preferably the furin cleavage site.

5. A diagnostic skin patch according to claim 2, wherein the isolated and purified Spike protein or one or more fragments thereof are produced in either of the following eukaryotic cells: mammalian cells, insect cells, wherein preferably the isolated and purified Spike protein or one or more fragments thereof is produced in a secreted form.

6. A diagnostic skin patch according to claim 2, wherein the recombinant SARS-CoV-2 virus protein antigen fragment is the receptor binding domain (RBD-protein) of the Spike protein.

7. A diagnostic skin patch according to claim 1, wherein one or more recombinant SARS-CoV-2 antigenic protein(s) are present in extracellular vesicles (ECVs), by which the protein(s) preserve the structure required for antigenicity.

8. A diagnostic skin patch according to claim 2, which contains an additional layer on the skin-contacting surface of the patch that facilitates the entry of the one or two recombinant, SARS-CoV-2 antigenic protein(s) to the epidermis, wherein preferably the skin-contacting additional layer contains a permeation enhancer device that enhances the entry of the antigenic protein(s) to the epidermis.

9. A diagnostic skin patch according to claim 1, wherein the matrix includes or consists of a glass fibre layer, onto which the antigen-storing compartment which includes one or more recombinant SARS-CoV-2 antigenic protein(s) dried on.

10. A diagnostic skin patch according to claim 1, in which the matrix contains a hydrogel, in which the antigen-storing compartment includes one or more recombinant, SARS-Co V-2 antigenic protein(s) embedded in the hydrogel.

11. A diagnostic skin patch according to claim 7, in which the permeation enhancer device contains microneedles penetrating the epidermis.

12. A method for the production of a diagnostic skin patch that is suitable for detecting the cellular immune response against SARS-CoV-2 virus, characterised by the following:
i) one or more SARS-CoV-2 antigenic proteins produced recombinantly, that preserve the native, antigenic structure
ii) a carrier layer is provided
iii) the carrier layer substantially contains:
a. an antigen-carrying compartment, that contains a matrix and one or more recombinant, SARS-CoV-2 antigenic proteins that can be detected by immune cells, and
b. a reference compartment, that contains the matrix but not the SARS-CoV-2 antigenic protein(s), wherein preferably the antigen-carrying compartment and the reference compartment are created in separated areas,
iv) the carrier layer is provided with attaching means to attach the skin patch on the human patient's skin surface, wherein preferably by attaching the skin patch on the human patient's skin, the antigenic protein enters the epidermis and it can contact with the human patient's immune cells against SARS-CoV-2 and trigger an immune response in the skin.

13. A method according to claim 12, characterised by the following:
the one or more SARS-CoV-2 antigenic protein is a recombinant Spike protein and/or one or more fragments thereof, having antigenic properties, wherein preferably
the recombinant Spike protein lacks the natural (furin) cleavage site and/or
the antigenic fragment produced is the Spike protein receptor binding domain (RBD-protein).

14. A method according to the claim 12, characterised by the following: in step i)
the recombinant Spike protein and/or one or more antigenic fragments thereof are expressed in eukaryotic cells selected from the following group:
mammalian cells, insect cells, in a secreted form,
the protein is purified from the cell culture media, while preserving the native structure required for antigenicity.

15. A method according to claim 12, characterised by the following:
the recombinant Spike protein and/or one or more antigenic fragments thereof are tagged with a tag that facilitates purification, preferably tagged with a histidine-tag and
the protein or protein fragment is isolated and purified from the cell culture media using the tag that facilitates purification, while preserving the native structure required for antigenicity.

16. A method according to claim 12, characterised by the following:
the recombinant Spike-protein and/or one or more antigenic fragments thereof are expressed in extracellular vesicles (ECV), thereby preserving the structure required for antigenicity.

17. A method according to claim 12, characterised by the following:
the antigen-carrying compartment and the reference compartment are created next to each other, but in well separated areas on the carrier layer.

18. A method according to claim 12, characterised by the following:
the antigen-carrying compartment is formed
by applying a glass fibre on the carrier layer as a matrix, onto which the one or more recombinant, SARS-CoV-2 antigenic proteins are dried, wherein the glass fibre layer is preferably a glass fibre filter disc, or
hydrogel is used as a matrix, in which one or more SARS-CoV-2 antigenic protein(s) are provided, the hydrogel is applied on the carrier layer
immediately before applying the patch or
formulated in a sealed form to retain moisture, or
the hydrogel is gently dried on the carrier layer.

19. A method according to claim 12, characterised by that when applying the skin patch, an additional layer is applied on the skin-contacting side of the patch to facilitate the entry of the recombinant SARS-CoV-2 antigenic proteins into the epidermis.

20. A method according to claim 12, characterised by that microneedles are formed on the on the layer contacting the skin.
